SEQUENCE LISTING (3 sequences)

SEQ ID NO: 1 - protein, 116 aa, Trypanosoma brucei
MQQPSQQQIQPIDSLSPFLGGKWWIRARVTDKSEIRTWNKPTSQGKLFSFTLIDESASIRATVFNEAVDMFNPLIVNGQVYYFSGGQVKNANRKFSNVNNDYELSFDNTCQISAAR

SEQ ID NO: 2 - protein, 116 aa, Trypanosoma cruzi
MQHPSNQQIQPIDSLTPFLGGKWWIRARVTDKSDVRTWNKPTSQGKLFSFTLIDESASIRATVFNDAVDLFNPLIVNGQVYYLSGGQVKNANRKFSNVNNDYELTFDSSSQISLAR

SEQ ID NO: 3 - protein, 116 aa, Leishmania sp.
MQQPGSHQIQPIDSLTPFLGGKWWIRARVTDKTDVRTWNKPTSQGKLFSFTLIDESAAIRATVFNDAVDTFEPLIVNGQVYYFSGGQVKNANRRFSNVNNDYELTFDRSSEIMLAR

(19) United States
(12) Patent Application Publication
(10) Pub. No.: US 2025/0188033 A1
(43) Pub. Date: Jun. 12, 2025

(54) INHIBITOR OF REPLICATION PROTEIN A1 OF TRYPANOSOMA BRUCEI AND METHOD OF USE THEREOF

(71) Applicants: Research Foundation of the City University of New York, New York, NY (US); Rutgers, The State University of New Jersey, New Brunswick, NJ (US); Universidad Nacional De San Martin, San Martin (AR)

(72) Inventors: Junyong Choi, Fresh Meadows, NY (US); Hee-Sook Kim, Harrison, NJ (US); Esteban Erben, Vicente Lopez (AR); Zakir Hossain, Jamaica, NY (US); Aditi Mukherjee, Newark, NJ (US); Diya Sharma, New York, NY (US)

(21) Appl. No.: 18/778,149

(22) Filed: Jul. 19, 2024

Related U.S. Application Data
(60) Provisional application No. 63/608,404, filed on Dec. 11, 2023.

(51) Int. Cl. C07D 211/96; A61K 31/445

(52) U.S. Cl. CPC C07D 211/96; A61K 31/445

(57) ABSTRACT

A composition of matter having a general structure of:

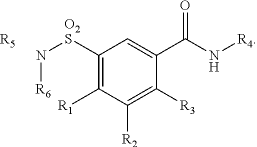

The composition selectively inhbits Replacement Proteins A1 (RPA1) of Trypanosoma brucei.

### CROSS-REFERENCE TO RELATED APPLICATIONS

This application claims priority to, and is a non-provisional of, U.S. Patent Application 63/608,404 (filed Dec. 11, 2023), the entirety of which is incorporated herein by reference.

### STATEMENT OF FEDERALLY SPONSORED RESEARCH OR DEVELOPMENT

This invention was made with government support under grant numbers SC2 GM130470 and R01 AI127562-01A1 awarded by the National Institute of Health. The government has certain rights in the invention.

### REFERENCE TO A SEQUENCE LISTING

This application contains a Sequence Listing in computer readable form. The computer readable form is incorporated herein by reference. The computer readable file is named Seqeuence.xml and was created on Jul. 19, 2024 (4 kB size).

### BACKGROUND OF THE INVENTION

Replication Protein A (RPA) complex is a broadly conserved complex comprised of the RPA1, RPA2 and RPA3 subunits. RPA protects genome integrity by binding ssDNA during DNA replication and repair. RPA1 is found in Trypanosoma brucei—the causative parasite of African trypanosomiasis.

Trypanosoma brucei is a protozoan parasite that causes African trypanosomiasis in humans (Human African Trypanosomiasis, HAT) in sub-Saharan Africa. This parasite also causes nagana, a form of trypanosomiasis found in cattle and other livestock. Transmission of the parasite occurs through biting by infected tsetse flies (Glossina spp.). African trypanosomiasis is one of twenty ‘neglected tropical diseases’ designated by the World Health Organization (WHO). Four drugs and a combination therapy have been used for the treatment of HAT: suramin and pentamidine target the early hemolymphatic stage of infection; melarsoprol, eflornithine, and a combination of eflornithine and nifurtimox are used for late meningoencephalitic stage infection in which parasites invade the central nervous system (CNS) after crossing the blood-brain barrier. These drugs have high rates of toxicity and are difficult to administer, and drug resistance quickly arises. Novel therapeutics are thus urgently needed.

The discussion above is merely provided for general background information and is not intended to be used as an aid in determining the scope of the claimed subject matter.

### SUMMARY

This disclosure provides a composition of matter having a general structure of:

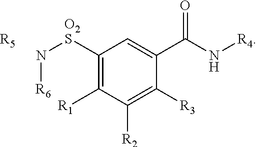

The composition selectively inhbits Replacement Proteins A1 (RPA1) of Trypanosoma brucei.

In a first embodiment, a composition of matter is provided. The composition has a structure as recited in Formula C-2:

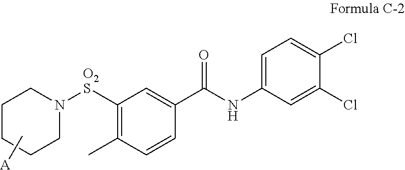

wherein A is selected from H, NH₂; NH-Boc (tert-butyloxycarbonyl); NH(SO₂)CH₃ and:

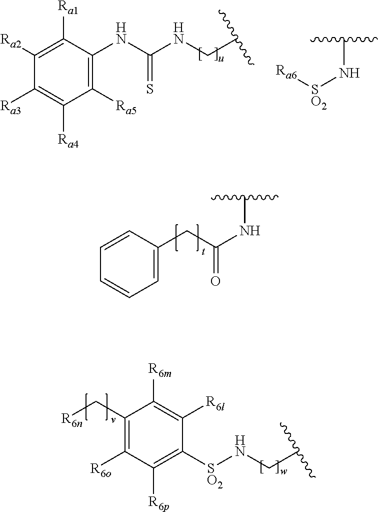

wherein t is 0, 1, 2 or 3; u is 0, 1, 2 or 3; Rₐ₁, Rₐ₂, Rₐ₃, Rₐ₄, Rₐ₅ are independently selected from H, F, Cl, OCH₃ and CF₃; Rₐ₆ is methyl, ethyl or propyl; v is 0, 1, 2 or 3; w is an integer from 0-6, inclusive; R₆ₗ, R₆ₘ, R₆ₙ, R₆ₒ and R₆ₚ are independently selected from H, F, Br, CH₃, CF₃ OBz and (CO)NPh where Ph is a 3,4-dichlorophenyl.

In a second embodiment, a composition of matter is provided. The composition has a structure as recited in Formula B:

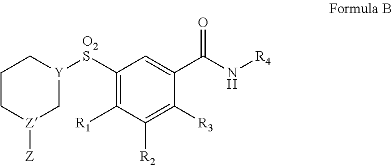

wherein R1, R₂ and R₃ are independently selected from H and a C1-C3 alkyl; R₄ is an organic group selected from:

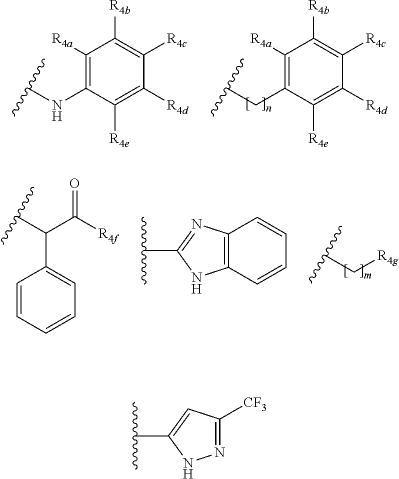

wherein R₄ₐ, R_{4b}, R_{4c}, R_{4d} and R₄ₑ are independently selected from H and Cl, n is 0, 1, 2 or 3, R_{4f} is NH₂, N(CH₃)₂, CO₂H, CO₂CH₃, R_{4g} is NH₂, N(CH₃)₂, OH and m is 0, 1, 2 or 3; Y is N or CH; Z′ is N or CH; and Z is selected from H, NH₂; NH-Boc (tert-butyloxycarbonyl); NH(SO₂)CH₃, a structure selected from

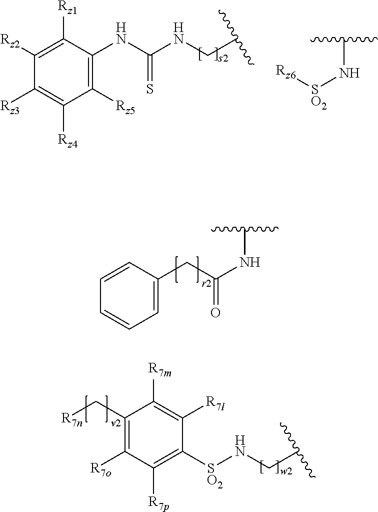

wherein r2 is 0, 1, 2 or 3; s2 is 0, 1, 2 or 3 and R_{z1}, R_{z2}, R_{z3}, R_{z4}, R_{z5} are independently selected from H, F, Cl, OCH₃ and CF₃; R_{z6} is methyl, ethyl or propyl; v2 is 0, 1, 2 or 3; w2 is an integer from 0-6 (inclusive); R₇ₗ, R₇ₘ, R₇ₙ, R₇ₒ and R₇ₚ are independently selected from H, F, Br, CH₃, CF₃, OBz and (CO)NPh where Ph is a 3,4-dichlorophenyl; or a pharmaceutically acceptable salt thereof.

In a third embodiment, a composition of matter is provided. The composition has a structure as recited in Formula C:

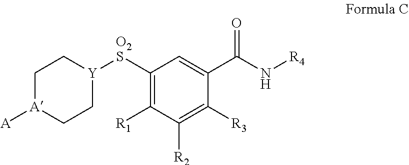

wherein R₁, R₂ and R₃ are independently selected from H and a C1-C3 alkyl; R₄ is an organic group selected from:

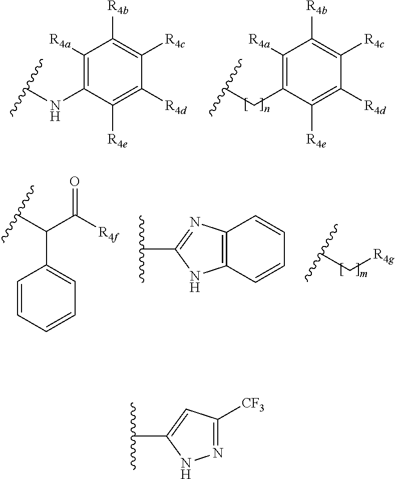

wherein R₄ₐ, R_{4b}, R_{4c}, R_{4d} and R₄ₑ are independently selected from H and Cl, n is 0, 1, 2 or 3, R_{4f} is NH₂, N(CH₃)₂, CO₂H, CO₂CH₃, R_{4g} is NH₂, N(CH₃)₂, OH and m is 0, 1, 2 or 3; Y is N or CH; A′ is N or CH; and A is selected from H, NH₂; NH-Boc (tert-butyloxycarbonyl); NH(SO₂)CH₃, a structure selected from

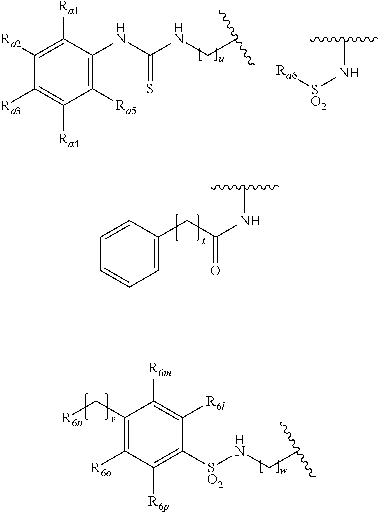

wherein t is 0, 1, 2 or 3; u is 0, 1, 2 or 3 and Rₐ₁, Rₐ₂, Rₐ₃, Rₐ₄, Rₐ₅ are independently selected from H, F, Cl, OCH₃ and CF₃; Rₐ₆ is methyl, ethyl or propyl; v is 0, 1, 2 or 3; w is an integer from 0-6 (inclusive); R₆ₗ, R₆ₘ, R₆ₙ, R₆ₒ and R₆ₚ are independently selected from H, F, Br, CH₃, CF₃ OBz and (CO)NPh where Ph is a 3,4-dichlorophenyl; or a pharmaceutically acceptable salt thereof.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1A show schematic diagrams of the three subunits that comprise human and T. brucei RPA complexes. Positions of amino-acid residues defining each domain are shown: DNA-Binding Domain, DBD; and Winged Helix, WH.

FIG. 1B is an X-ray co-crystal structure of HsDBD-F (light blue) in complex with ATRIP derived peptide (green) (PDB code: 4NB3) and a 3-D homology model of TbRPA1 DBD-A (red) interacting with ssDNA.

FIG. 1C is an induced-fit docking model showing the binding pose of JC-229 in the ssDNA-binding pocket of TbRPA1 DBD-A (cartoon (left) and sphere (middle) models shown), and chemical structure of JC-229.

FIG. 1D depicts one synthetic scheme for JC-229.

FIG. 1E is a graph showing inhibition of T. brucei cell growth by JC-229. Wild-type (WT) T. brucei cells were treated with 50 μM JC-229 for 3 days and viability was determined using the ALAMARBLUE® assay.

FIG. 1F is a dose-dependent growth inhibition curve of T. brucei by JC-229. T. brucei cells were treated with increasing concentrations of JC-229 (0.5 to 50 M) for 72 hours and viability was determined with the ALAMARBLUE® assay. A standard 4-Parameter Logistic (4 PL) curve from Prism software generated the EC50 value. All growth experiments were performed in triplicate.

FIG. 2A is a graph showing TbRPA1 is essential for T. brucei cell viability. TbRPA1 RNAi depletion was induced with addition of tetracycline and cell growth was monitored by counting cells every 12 hours for 48 hours. WT was used as a control. Error bars indicate standard deviation (n=3). Two-tailed t-test was performed and statistically significant results were indicated with ****P<0.0001.

FIG. 2B is a graph showing viability of TbRPA1-depleted trypanosome cells. Cells from each time point in FIG. 2A were examined for viability using the ALAMARBLUE® assay. Percent viability compared to non-depleted cells was determined and plotted. Error bars indicate standard deviation (n=3). Two-tailed t-test was performed and statistically significant results were indicated with ****P<0.0001.

FIG. 2C shows immunoblot controls presenting the changes in protein levels for TbRPA1, γH2A, VSG3, and Tubulin at each time point after depletion. Tubulin serves as a loading control.

FIG. 2D depicts graphs with cell-cycle profiles of TbRPA1-depleted cells. Fixed cells were stained with Propidium Iodide (PI) and analyzed by flow cytometry.

FIG. 2E shows a DNA synthesis assay by BrdU pulse labeling. WT and TbRPA1 KD cells were pulse-labeled with 500 μM BrdU and fixed. Fixed cells were then stained with PI (bulk DNA) and anti-BrdU-Alexa 488 antibody (newly synthesized DNA) and analyzed by flow cytometry.

FIG. 3A depicts cell-cycle profiles of JC-229-treated T. brucei cells. WT T. brucei cells were treated with 10 μM of JC-229 and collected at indicated time points. Cells were fixed, stained with PI, and analyzed by flow cytometry.

FIG. 3B shows a DNA synthesis assay by BrdU pulse labeling. T. brucei cells treated with 10 μM JC-229 for 0, 8, 16 and 24 hours were pulse-labeled with BrdU as shown in FIG. 2E. Cells were fixed, stained with PI and anti-BrdU-Alexa 488 antibody, and then analyzed by flow cytometry.

FIG. 3C depicts immunoblots showing the levels of TbRPA1, γH2A, VSG3, and Tubulin proteins in cells treated with JC-229 for indicated hours. Tubulin serves as a loading control.

FIG. 3D shows quantification of protein levels in immunoblots. Signal intensities of protein bands were quantified using ImageJ software, normalized to those of tubulin bands. Fold changes relative to the untreated samples were plotted. Error bars indicate standard deviation (n=3).

FIG. 3E illustrates a microscopic analysis of JC-229 treated T. brucei cells. Cells collected at indicated time points were fixed in 1% paraformaldehyde for 10 min and stained with DAPI. Images were obtained using an EVOS M5000 microscope. Scale bar, 10 μm.

FIG. 3F is a graph showing cytotoxicity of JC-229 on T. brucei and two human cell lines (HeLa and HEK293). Dose-response curve was obtained from cells treated with increasing concentrations of JC-229 (0.5, 1.25, 2.5, 5, 10, 20, 30, 40 and 50 μM) for 72 hours. Experiments were performed in triplicate and EC₅₀ values were obtained from GraphPad Prism software (inhibitor vs. normalized response with variable slopes).

FIG. 4A shows increasing concentrations (0.05, 0.2, 1.6, 3.2, 6.25, 12.5, 25, 50 and 100 nM) of TbRPA1 DBD-AB protein were incubated with 2 nM oligo dT32 labeled with 5′IRDye (5′IRDye800-dT₃₂) and the protein-ssDNA complex was analyzed by EMSA (left). Serial dilutions (0.625, 1.25, 2.5, 5, 10, 20, 40 and 80 μM) of JC-229 were pre-incubated with 12.5 nM TbRPA1 DBD-AB protein, then incubated with 5′IRDye800-dT₃₂. Inhibition of binding was visualized by EMSA (right).

FIG. 4B shows the results of an assay like that of FIG. 4A with HsRPA1 DBD-AB.

FIG. 4C shows the results of an assay like that of FIG. 4A with TbRPA complex.

FIG. 4D shows the results of an assay like that of FIG. 4A with HsRPA complex.

FIG. 4E and FIG. 4F show SDS-PAGE with purified fractions used for EMSA assays. All EMSA experiments were performed in triplicate.

FIG. 5A are graphs demonstrating TbRPA1 DBD-AB proteins were 2-fold serially diluted starting with a 1000 nM concentration and then incubated with 10 nM oligo dT₃₂ labeled with 5′Cy5 (5′Cy5-dT₃₂). Binding was analyzed by MST (left). Serial dilutions of JC-229 were pre-incubated with 30 nM TbRPA1 DBD-AB protein and then with 10 nM 5′Cy5-dT₃₂. Inhibition of binding was analyzed by MST (right).

FIG. 5B are graphs showing HsRPA1 DBD-AB protein was examined as in FIG. 5A by MST.

FIG. 5C are graphs showing recombinant TbRPA complex was 2-fold serially diluted from 400 nM concentration and these were incubated with 10 nM 5′Cy5-dT₃₂. Binding was analyzed by MST (left). Serial dilutions of JC-229 were pre-incubated with 25 nM TbRPA complex and then with 10 nM 5′Cy5-dT₃₂. Inhibition of binding was analyzed by MST (right).

FIG. 5D are graphs showing HsRPA complex was examined as in FIG. 5C. All experiments were performed in triplicate. Statistical analysis and plotting were performed with GraphPad Prism software. K_{d} and IC₅₀ values were obtained with a one site binding total and standard 4-PL curve respectively.

FIG. 6A shows the results of an EMSA performed on recombinant TcRPA1 DBD-AB as described in FIGS. 4A-4F. Left: ssDNA-binding activity of TcRPA1 DBD-AB. Right: Inhibition of ssDNA-binding activity of TcRPA1 DBD-AB by JC-229.

FIG. 6B shows the results of an EMSA assay on recombinant LmexRPA1 DBD-AB. Left: ssDNA-binding activity of LmexRPA1 DBD-AB. Right: Inhibition of ssDNA-binding activity of LmexRPA1 DBD-AB by JC-229.

FIG. 6C shows the results of a MST assay performed on recombinant TcRPA1 DBD-AB as described in FIGS. 5A-5D. Left: ssDNA-binding activity of TcRPA1 DBD-AB. Right: Inhibition of ssDNA-binding activity of TcRPA1 DBD-AB by JC-229 (30 nM protein used).

FIG. 6D shows the results of a MST assay performed on recombinant LmexRPA1 DBD-AB as in (C). Left: ssDNA-binding activity of LmexRPA1 DBD-AB. Right: Inhibition of ssDNA-binding activity of LmexRPA1 DBD-AB by JC-229. All experiments were performed in triplicate. Statistical analysis and plotting of MST data were performed with GraphPad Prism software. K_{d} and IC₅₀ values were obtained with a one site binding total and standard 4-PL curve respectively.

FIG. 6E illustrates an SDS-PAGE gel showing purified fractions used for EMSA and MST assays.

FIG. 7A is a sequence comparison of DBD-A domains of Tb, Tc and LmexRPA1. S105 of TbRPA1 DBD-A and T105 of Tc and LmexRPA1 DBD-A are shown.

FIG. 7B depicts binding pose of JC-229 with the surface of S105 in DBD-A of TbRPA1 is shown in (a). Binding pose of JC-229 with T105 in DBD-A of TcRPA1 are shown (b: ribbon and c: sphere model).

FIG. 7C depicts an SDS-PAGE gel showing purified fractions of TbRPA1 DBD-AB S105T and TcRPA1 DBD-AB T105S mutant proteins.

FIG. 7D depicts EMSA that was performed as described in FIGS. 4A-4F. Left: ssDNA-binding activity of TbRPA1 DBD-AB S105T mutant. Right: Inhibition of ssDNA-binding activity of Tb S105T mutant by JC-229.

FIG. 7E are graphs showing the results of a MST assay performed on recombinant TbRPA1 DBD-AB S105T as described in FIGS. 5A-5D. Left: ssDNA-binding activity of TbRPA1 DBD-AB S105T mutant. Right: Inhibition of ssDNA binding activity of Tb S105T mutant by JC-229 (30 nM protein used).

FIG. 7F shows the results of an EMSA assay. Left: ssDNA binding activity of TcRPA1 DBD-AB T105S mutant. Right: Inhibition of ssDNA-binding activity of Tc T105S mutant by JC-229.

FIG. 7G are graphs showing the results of a MST assay. Left: ssDNA-binding activity of TcRPA1 DBD-AB T105S mutant. Right: Inhibition of ssDNA-binding activity of Tc T105S mutant by JC-229 (30 nM protein used). MST data of WT TbRPA1 DBD-AB from FIG. 5A and WT TcRPA1 DBD-AB from FIG. 6C are overlaid for comparison (FIG. 7E, FIG. 7G). All EMSA and MST experiments were performed in triplicate. Statistical analysis and plotting of MST data were performed with GraphPad Prism software. K_{d} and IC₅₀ values were obtained with a one site binding total and standard 4-PL curve respectively.

FIG. 8 is a graph depicting percent inhibition of select JC compounds.

### DETAILED DESCRIPTION OF THE INVENTION

This disclosure provides an inhibitor that targets RPA1. The inhibitor is specifically toxic to T. brucei cells and only mildly toxic to human cells. The inhibitor treatment mimics the effects of TbRPA1 depletion, including DNA replication inhibition and DNA damage accumulation. In-vitro ssDNA-binding assays demonstrate that the inhibitor inhibits the activity of TbRPA1, but not the human ortholog. Indeed, despite the high sequence identity of T. cruzi and Leishmania RPA1, the disclosed inhibitor only impacts the ssDNA-binding activity of TbRPA1. Site-directed mutagenesis confirms that the DNA-Binding Domain A (DBD-A) in TbRPA1 contains an inhibitor binding pocket. Residue Serine 105 determines specific binding and inhibition of TbRPA1 but not T. cruzi and Leishmania RPA1.

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre and post natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Salt: As used herein, the term “salt” refers to a pharmaceutically acceptable salt of a composition, such as a sodium salt, a sulfate salt, an acetate salt, a phosphate salt, a diphosphate salt, a chloride salt, a potassium salt, a maleate salt, a trifluoroacetic acid (TFA) salt, a calcium salt, a citrate salt, a mesylate salt, a nitrate salt, a tartrate salt, an aluminum salt, a gluconate salt, and the like.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. Those of ordinary skill in the art will appreciate that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Administering: As used herein, the term “administer,” “administering” or grammatical equivalent, refers to the method of treating a patient and includes, for example, oral administering, intravenous administering, topical administering, and the like.

Eukaryotic RPA subunits, including the human ortholog, contain six ssDNA-binding domains (DBDs): four in the largest subunit RPA1 (DBD-F, A, B and C in tandem) and one each in RPA2 (DBD-D) and RPA3 (DBD-E). All DBDs have a specific tertiary structure called the oligosaccharide/oligonucleotide-binding (OB) fold, which is known to bind ssDNA. The central region of the RPA1 subunit, DBD-A and B, dominates during ssDNA binding, whereas the N-terminal domain of RPA1 (DBD-F or RPA1-N) is involved in protein-protein interactions (PPI).

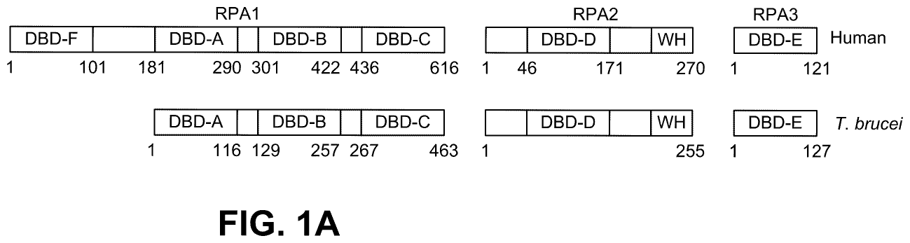

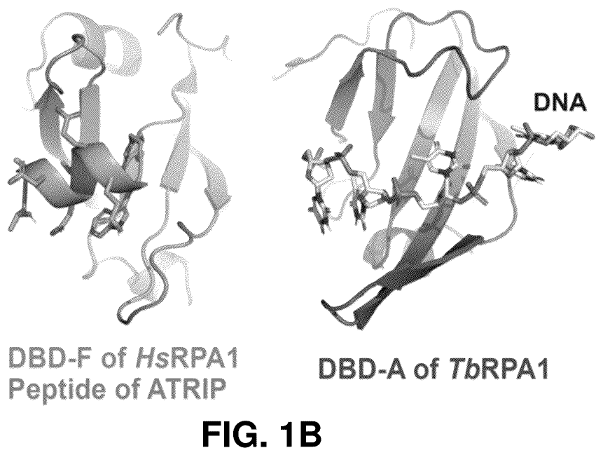

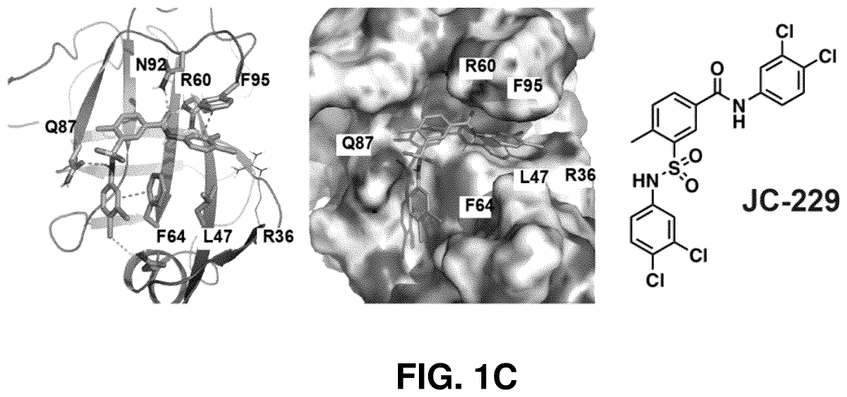

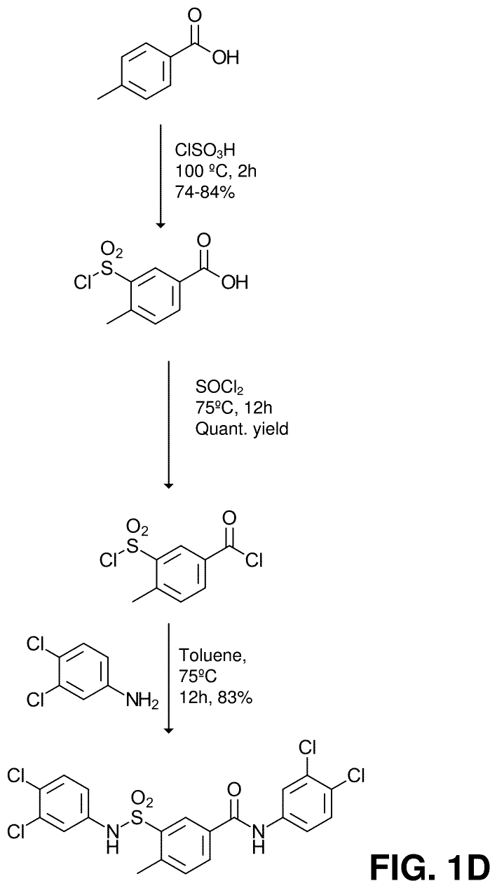

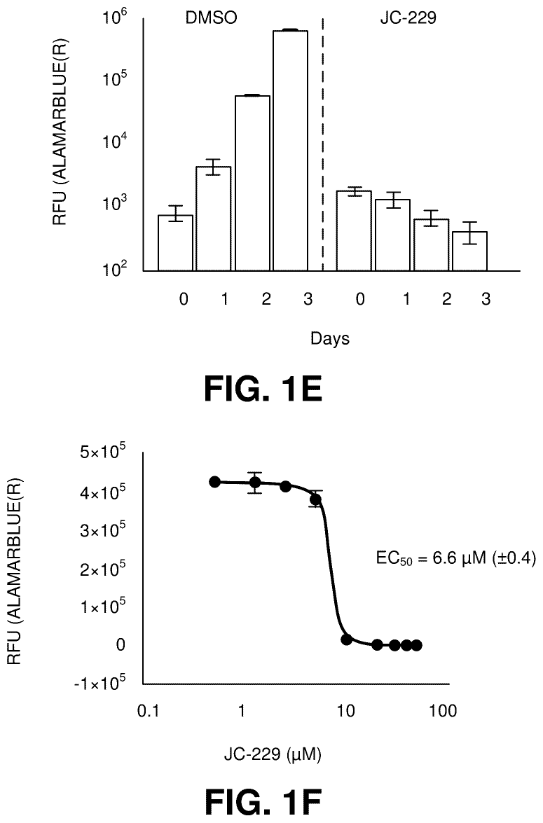

All three subunits of RPA are present in trypanosomatids, including T. brucei, T. cruzi, and Leishmania spp. Trypanosomatid RPA1 sequences and domain structures are mostly conserved as in mammals, except that the parasite proteins lack the N-terminal 180 amino acids of the DBD-F domain (FIG. 1A). Molecular functions of RPA in these parasites have not been thoroughly explored and only limited information is available to date. T. brucei RPA is the least studied among these three trypanosomatids. T. brucei RPA1 forms nuclear foci in response to DSBs or in replication-defective mutants. Although available data are limited, several unique features of trypanosome RPA1 offer opportunities for selective therapeutic development, including the lack of DBD-F in RPA1, RPA nuclear export during differentiation, and additional roles in telomere protection.

Data provided in this disclosure demonstrates T. brucei replication proteins are potential therapeutic targets for the treatment of African trypanosomiasis. The phenotypes associated with the depletion of TbRPA1 protein were characterized and the effects of repositioned HsRPA1 PPI inhibitors on T. brucei cell growth were examined. An in-house 3-D structural model of TbRPA1 has been utilized to discover inhibitors possessing specific inhibitory effects on TbRPA1, but not on human RPA1. Site-directed mutagenesis driven by molecular modeling confirmed that ssDNA and the disclosed inhibitors share a binding site in DBD-A of TbRPA1. In particular, the S105T mutant completely abolished the inhibitory effect of the disclosed inhibitor without altering the ssDNA-binding capacity of TbRPA1. These data point toward a therapeutic that targets TbRPA1 while having minimal cytotoxic effects on human cells.

In one embodiment, the inhibitor has a structure as shown in Formula A

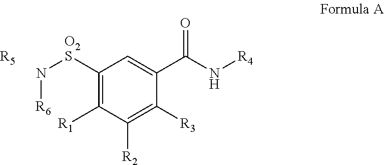

In Formula A, R₁, R₂ and R₃ are independently selected from H and an alkyl. The alkyl may be, for example, methyl, ethyl or propyl (e.g. n-propyl, 2-propyl).

R₄ is an organic group selected from:

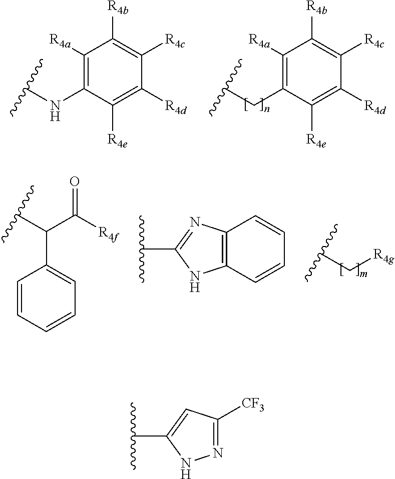

wherein R₄ₐ, R_{4b}, R_{4c}, R_{4d} and R₄ₑ are independently selected from H and Cl, n is 0, 1, 2 or 3, R_{4f} is NH₂, N(CH₃)₂, CO₂H, CO₂CH₃, R_{4g} is NH₂, N(CH₃)₂, OH and m is 0, 1, 2 or 3. In one embodiment, at least two of R₄ₐ, R_{4b}, R_{4c}, R_{4d} and R₄ₑ are Cl and the remainder are H. In one embodiment, two of R₄ₐ, R_{4b}, R_{4c}, R_{4d} and R₄ₑ are Cl and three are H. In one embodiment, R₄ₐ is H, R₄ₑ is H, R_{4c} is Cl, R_{4d} is Cl and R₄ₑ is H.

R₅ and R₆ are independently selected from H, benzyl (Bz) and:

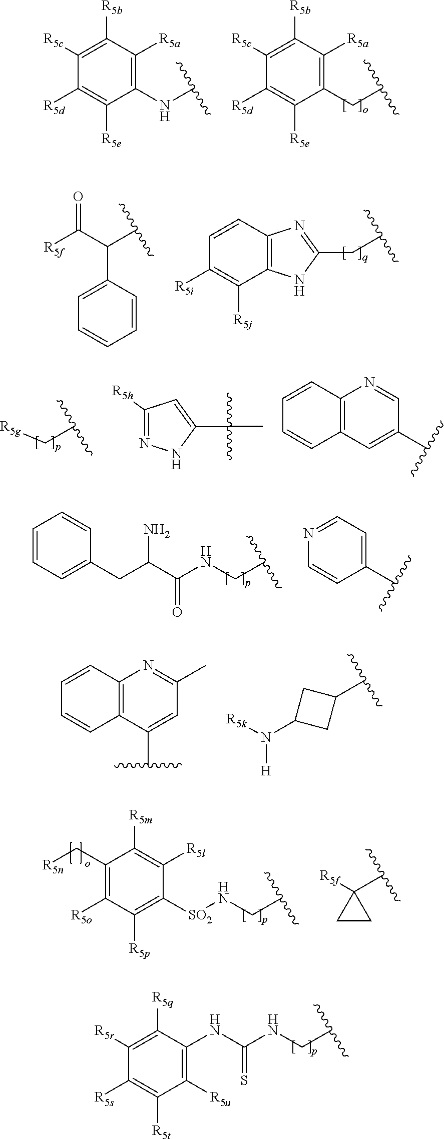

wherein R₅ₐ, R_{5b}, R_{5c}, R_{5d} and R₅ₑ are independently selected from H, Cl, OH, CN, NH₂, NO₂, CCH and (CO)CH₃; o is 0, 1, 2 or 3; R_{5f} is NH₂, N(CH₃)₂, CO₂H or CO₂CH₃; and R_{5g} is NH₂, NH-Boc, NH(CO)CH₃, NH(CO)Ph, NH(CO)CH₂Ph, NH(CO)CH₂CH₂Ph, N(CH₃)₂, OH, (CH₂)₂OH, CH₃, or Ph; p is an integer from 0-6 (inclusive); R₅ₕ is H, CF₃ or C(CH₃)₂; R₅ᵢ and R₅ⱼ are independently selected from H and F an q is 0, 1, 2, or 3; R₅ₖ is NH₂, NH-Boc, R₅ₗ, R₅ₘ, R₅ₙ, R₅ₒ and R₅ₚ are independently selected from H, F, Br, CH₃, CF₃ OBz, (CO)NPh where Ph is a 3,4-dichlorophenyl; R_{5q}, R₅ᵣ, R₅ₛ, R₅ₜ, R₅ᵤ are independently selected from H, OCH₃, F, Cl and CF₃. In one embodiment, at least two of R₅ₐ, R_{5b}, R_{5c}, R_{5d} and R₅ₑ are Cl and the remainder are H. In one embodiment, two of R₅ₐ, R_{5b}, R_{5c}, R_{5d} and R₅ₑ are Cl and three are H. In one embodiment, R₅ₐ is H, R_{5b} is H, R_{5c} is Cl, R_{5d} is Cl and R₅ₑ is H. “Boc” is tert-butyloxycarbonyl.

In one embodiment, R₁ is CH₃, R₂ is H, R₃ is H, R₄ and R₅ are 3,4-dichlorophenyl and the resulting inhibitor is:

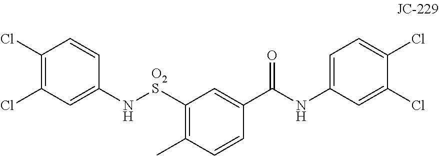 [SMILES: Cc1ccc(C(=O)Nc2ccc(Cl)c(Cl)c2)cc1S(=O)(=O)Nc1ccc(Cl)c(Cl)c1]

One synthesis of JC-229 is shown in FIG. 1D.

In one embodiment, the inhibitor has a structure as shown in Formula A-1:

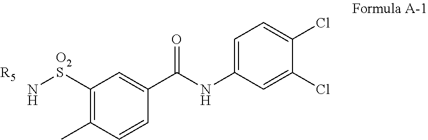

wherein R₅ is selected as recited with respect to Formula A.

In one embodiment, the inhibitor has a structure as shown in Formula B

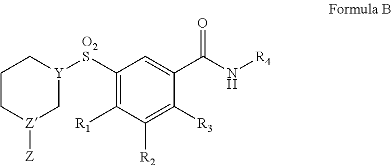

In Formula B, R₁, R₂ and R₃ are independently selected as recited with respect to Formula A. R₄ is the organic group as recited with respect to Formula A. Y is N or CH. Z′ is N or CH.

Z is selected from H, NH₂; NH-Boc; Boc (provided Z is N); NH(SO₂)CH₃ and:

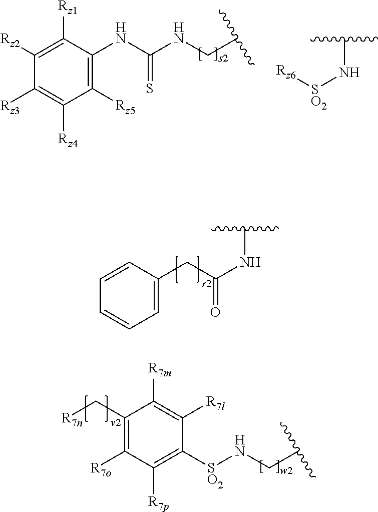

wherein r2 is 0, 1, 2 or 3; s2 is 0, 1, 2 or 3 and R_{z1}, R_{z2}, R_{z3}, R_{z4}, R_{z5} are independently selected from H, F, Cl, OCH₃ and CF₃; R_{z6} is methyl, ethyl or propyl (e.g. n-propyl or i-propyl); v2 is 0, 1, 2 or 3; w2 is an integer from 0-6 (inclusive); R₇ₗ, R₇ₘ, R₇ₙ, R₇ₒ and R₇ₚ are independently selected from H, F, Br, CH₃, CF₃, OBz, (CO)NPh where Ph is a 3,4-dichlorophenyl.

In one embodiment, R₁ is CH₃, R₂ is H, R₃ is H, R₄ is 3,4-dichlorophenyl, Y is N, Z′ is CH and Z is NH₂, such that the resulting inhibitor is:

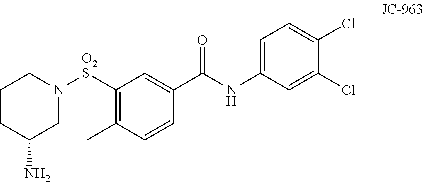 [SMILES: Cc1ccc(C(=O)Nc2ccc(Cl)c(Cl)c2)cc1S(=O)(=O)N1CCC[C@@H](N)C1]

In one embodiment, the inhibitor has a structure as shown in Formula B-1:

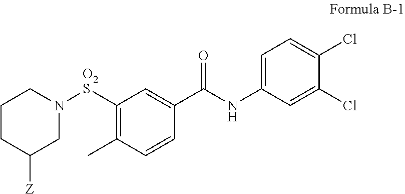

wherein Z is selected as recited with respect to Formula B.

In one embodiment, the inhibitor has a structure as shown in Formula C:

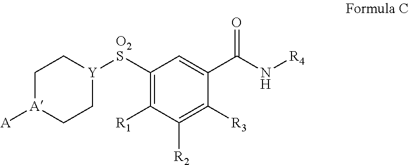

In Formula C, R₁, R₂ and R₃ are independently selected as recited with respect to Formula A. R₄ is the organic group as recited with respect to Formula A. Y is N or CH. A′ is N or CH.

A is selected from H, NH₂; NH-Boc; NH(SO₂)CH₃ and:

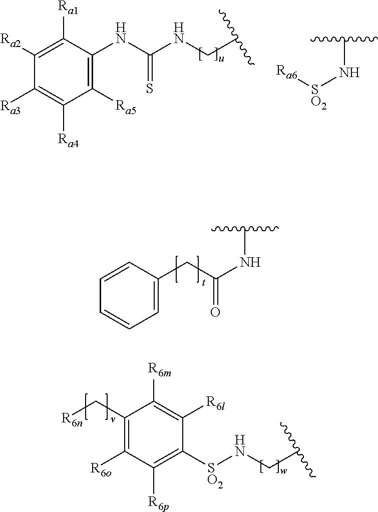

wherein t is 0, 1, 2 or 3; u is 0, 1, 2 or 3 and Rₐ₁, Rₐ₂, Rₐ₃, Rₐ₄, Rₐ₅ are independently selected from H, F, Cl, OCH₃ and CF₃; Rₐ₆ is methyl, ethyl or propyl (e.g. n-propyl or i-propyl); v is 0, 1, 2 or 3; w is an integer from 0-6 (inclusive); R₆ₗ, R₆ₘ, R₆ₙ, R₆ₒ and R₆ₚ are independently selected from H, F, Br, CH₃, CF₃ OBz, (CO)NPh where Ph is a 3,4-dichlorophenyl.

In one embodiment, R₁ is CH₃, R₂ is H, R₃ is H, R₄ is 3,4-dichlorophenyl, Y is N, A′ is CH and A is:

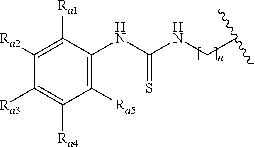

wherein u is 0, Rₐ₁, Rₐ₂, Rₐ₄ and Rₐ₅ are all H, and Rₐ₃ is F, such that the resulting inhibitor is:

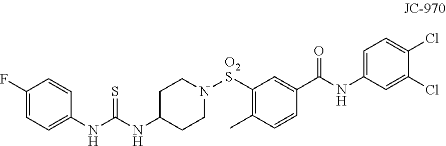 [SMILES: Cc1ccc(C(=O)Nc2ccc(Cl)c(Cl)c2)cc1S(=O)(=O)N1CCC(NC(=S)Nc2ccc(F)cc2)CC1]

In one embodiment, the inhibitor has a structure as shown in Formula C-1:

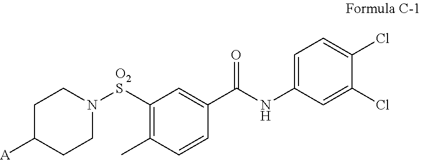

wherein A is selected as recited with respect to Formula C.

In one embodiment, the inhibitor has a structure as shown in Formula C-2:

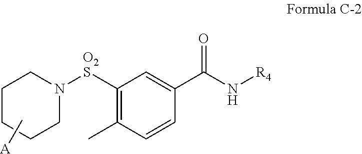

wherein A is selected as recited with respect to Formula C.

In one embodiment, the inhibitor has a structure as shown in Formula C-3:

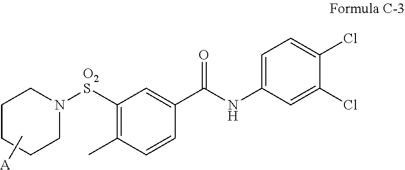

wherein A is selected as recited with respect to Formula C.

In one embodiment, the inhibitor has a structure as shown in Formula C-4:

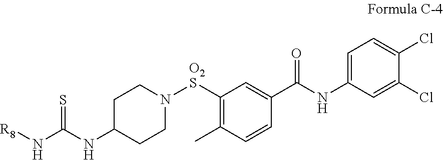

wherein R₈ is

In one embodiment, the inhibitor has a structure as shown in Formula D:

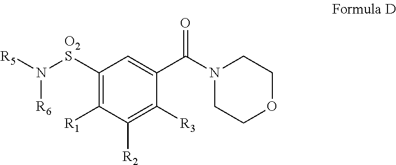

In Formula D, R₁, R₂ and R₃ are independently selected as recited with respect to Formula A. R₅ and R₆ are independent selected as recited with respect to Formula A.

In one embodiment, the inhibitor has a structure as shown in Formula E:

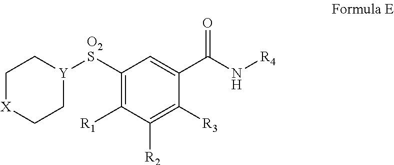

In Formula E, R₁, R₂ and R₃ are independently selected as recited with respect to Formula A. Y is N or CH. X is 0, CF2, CHNH2, NH, N-Boc, NCH3, CH—NH-Boc, or:

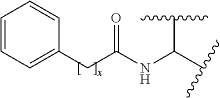

wherein x is 0, 1, 2 or 3.

In one embodiment, the inhibitor has a structure as shown in Formula F:

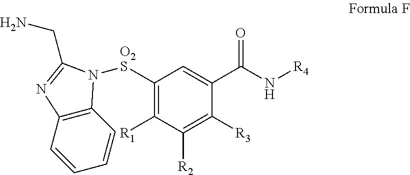

wherein R₁, R₂, R₃ and R₄ are independently selected as recited with respect to Formula A.
Inhibitors of T. brucei Cell Growth and Interaction with TbRPA1.

RPA1 has three ssDNA-binding domains (DBD-A, DBD-B, and DBD-C) and an N-terminal protein-protein interaction domain (DBD-F) that is conserved in humans and eukaryotic model systems. However, T. brucei RPA1 lacks the N-terminal PPI domain, although the three ssDNA-binding domains are present (FIG. 1A). Because no NMR or crystallographic TbRPA1 structures are currently available, a 3-D structural model of TbRPA1 was generated by applying homology modeling in the SWISS-MODEL server. RPA1 has high sequence identity between species: HsRPA1 and TbRPA1 have 54% sequence identity in DBD-A and 34% in DBD-B. In addition, the DBD domains in RPA1 share similar tertiary structures, since all DBDs, including DBD-F, contain an OB fold. As shown in FIG. 1B, the tertiary structure of HsRPA1 DBD-F (blue) resembles that of TbRPA1 DBD-A (red): HsRPA1 DBD-F harbors a binding pocket for ATRIP peptide-37 (green) (PDB code: 4NB3) and TbRPA1 DBD-A binds ssDNA. Interestingly, ATRIP peptide-37 interacts with HsRPA1 DBD-F and DBD-AB with similar binding affinities (K_{d}=7.4 μM and 12 μM, respectively). Thus, inhibitors that occupy the ATRIP-binding pocket of HsRPA1 DBD-F are believed to also bind the TbRPA1-DBD-AB, blocking the interaction between TbRPA1 and ssDNA.

Several analogs of known HsRPA1 inhibitors were tested and JC-229 was identified as a potential inhibitor of TbRPA1. T. brucei cells were treated with 50 μM JC-229 and cell growth was monitored for 72 hours using cell counting and the ALAMARBLUE® cell viability assay. While no growth inhibition was observed in T. brucei cells treated with DMSO (vehicle control), JC-229 treatment abolished parasite proliferation (FIG. 1E, FIG. 1F). To obtain a Half Maximal Effective Concentration (EC₅₀) of JC-229, T. brucei cells were treated with serial dilutions of JC-229 (from 0.5 μM to 50 μM) and determined viability using ALAMARBLUE® assay, thus obtaining an EC₅₀ of 6.6 μM (±0.4).

Induced fit docking with a homology model confirmed that JC-229 occupies the expected ssDNA-binding pocket of TbRPA1 DBD-A (FIG. 1C). Particularly, the sulfonamide and amide units of JC-229 form hydrogen bond interactions with Q87 and N92/R60 respectively, and two di-chlorophenyl rings have π-π stacking interactions with F64 and F95 of TbRPA1 DBD-A. TDRL-505, a known inhibitor of HsRPA1 ssDNA-binding activity, was also examined but no effect on T. brucei proliferation was observed, even at 60 μM concentration.

Depletion of T. brucei RPA1 Leads to Cell Lethality and Inhibition of DNA Replication

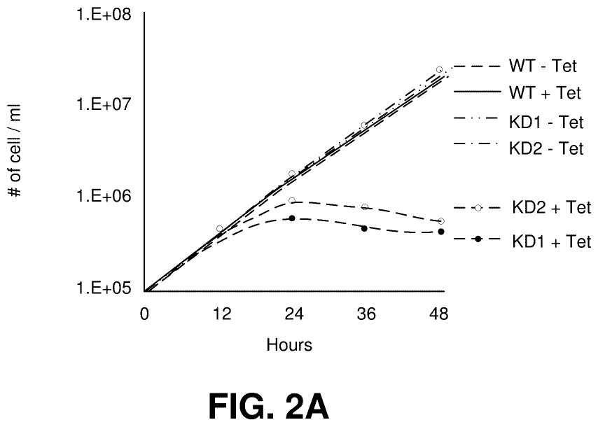

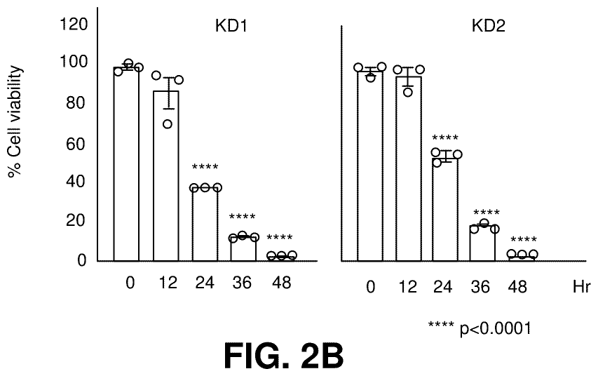

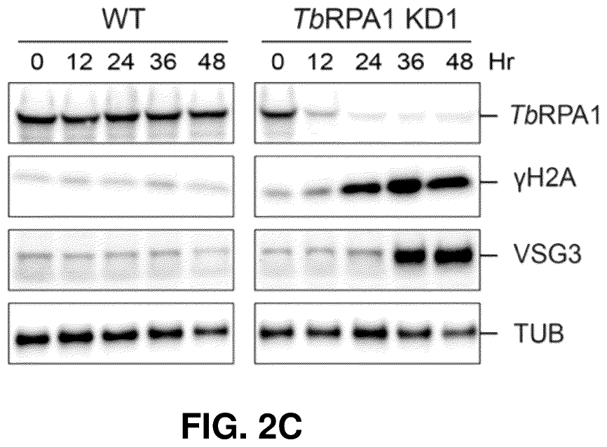

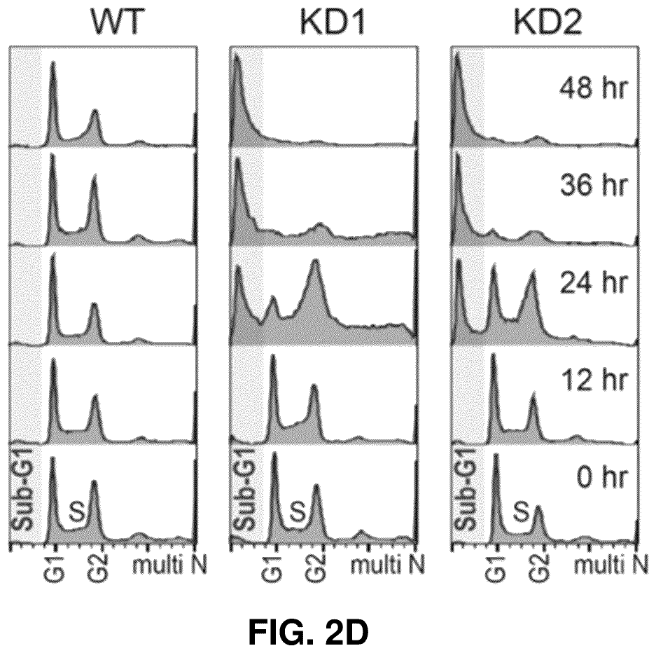

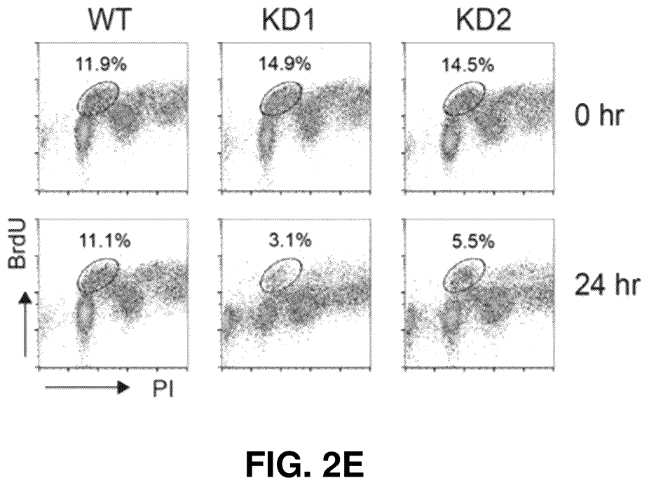

To see whether JC-229 impacts TbRPA1 function, cellular phenotypes following TbRPA1-depletion were examined. TbRPA1 knockdown (KD) was achieved by the expression of dsRNA targeting the TbRPA1 transcript under tetracycline control (Tet-On system). Stagnation of growth was observed 24-hour after induction of RNAi in two independent KD cell lines. The number of cells decreased after 36 and 48 hours of KD induction (FIG. 2A). To determine the percent viability of KD cells at each time point, an ALAMARBLUE® assay was performed (FIG. 2B). The majority of cells detected after 36 hours were dead or dying; 12% (KD1) and 17% (KD2) of cells were alive after 36 hours; only 2% (KD1) and 3% (KD2) of cells were living after 48 hours. These data confirm that TbRPA1 is essential for cell viability. Depletion of TbRPA1 protein in KD1 and KD2 cell lines was confirmed by immunoblotting (FIG. 2C).

TbRPA1 forms nuclear foci in response to double strand breaks or replication stress. Without wishing to be bound to any particular theory, DNA damage is believed to accumulate in the absence of TbRPA1. To test this idea, phosphorylated H2A levels (γH2A, the equivalent of γH2AX in mammals) were examined, which is a marker of DSB⁵⁰. Levels of γH2A were indeed increased after TbRPA1 depletion (FIG. 2C).

The cell surface of T. brucei is densely coated with a single species of Variant Surface Glycoprotein (VSG). Although the T. brucei genome carries 2,000 VSG alleles, the parasite expresses only one VSG allele per cell at any given time and suppresses the expression of the other alleles (VSG silencing). T. brucei has mechanisms to switch the expressed VSG, which removes the old VSG coat and presents a new VSG on the surface. This VSG switching mechanism, known as antigenic variation, allows the parasite to escape host immune recognition and killing. VSG switching events can be triggered by a DSB. Because DSBs accumulate in the absence of TbRPA1, VSG switching rate may increase in KD cells. Switching allows the expression of transcriptionally silent VSGs, which can be detected by immunoblotting. Antibodies against a silent VSG, VSG3 were used and an increase in VSG3 protein level was detected (FIG. 2C). However, since de-repression of VSG silencing would also increase VSG3 protein levels, VSG3 protein upregulation in TbRPA1-depleted cells could be due to an increased rate of VSG switching and disruption of VSG silencing. In either case, the upregulation of VSG3 protein can be used as a readout to determine whether inhibitors are causing TbRPA1-specific defects.

RPA is required for DNA replication initiation and elongation. Removal of TbRPA1 protein was anticipated to inhibit DNA replication and disrupt normal cell-cycle progression. TbRPA1-depleted cells were fixed and stained with Propidium Iodide (PI) to stain bulk DNA and analyze cells by flow cytometry. Two clear aberrations were observed in the absence of TbRPA1: the accumulation of G2-phase cells followed by an increase in sub-G1 cells (<2C DNA content) (FIG. 2D). At 48 hours after KD induction, ˜60% cells showed <2C DNA content. In the absence of TbRPA1, cells may arrest at G2/M phase initially and die eventually, thus generating a sub-G1 population. DNA synthesis was also compromised in the absence of TbRPA1, as expected (FIG. 2E). The number of S phase cells incorporating BrdU, a dT analog that is incorporated into newly synthesized DNA during S phase, was reduced after the depletion of TbRPA1.

JC-229 Treatment Resembles TbRPA1 Depletion and has Selective Toxicity Against T. brucei

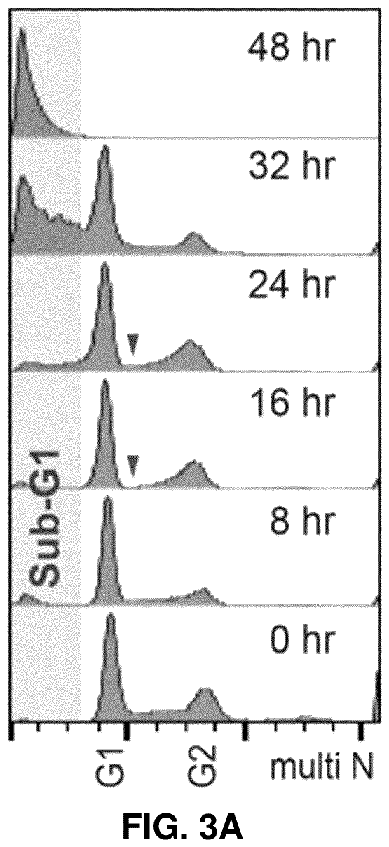

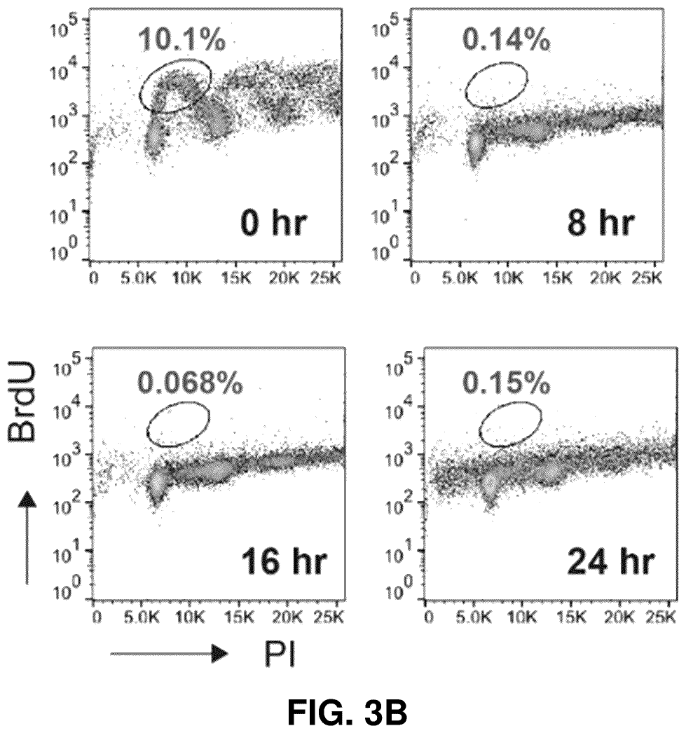

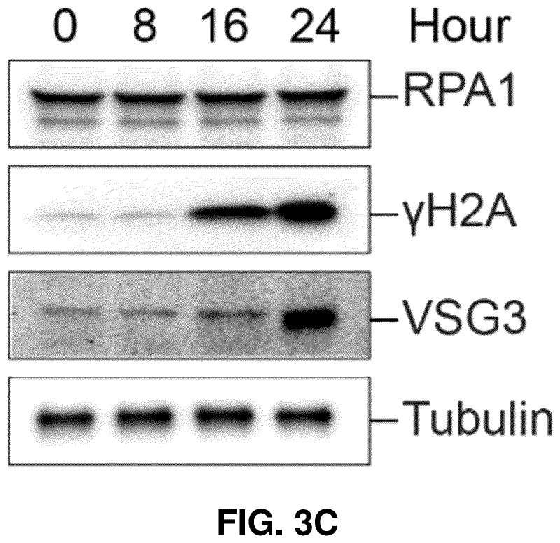

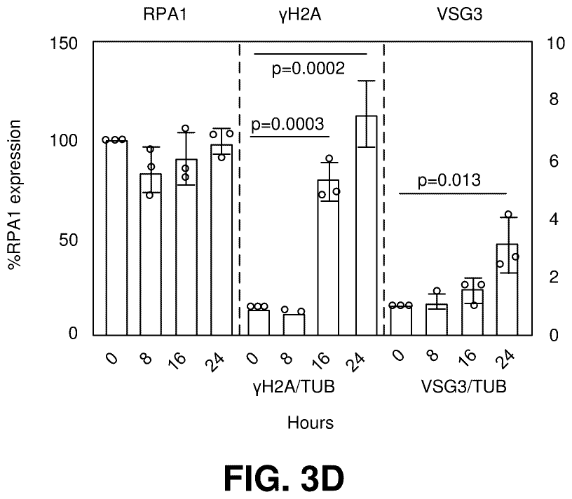

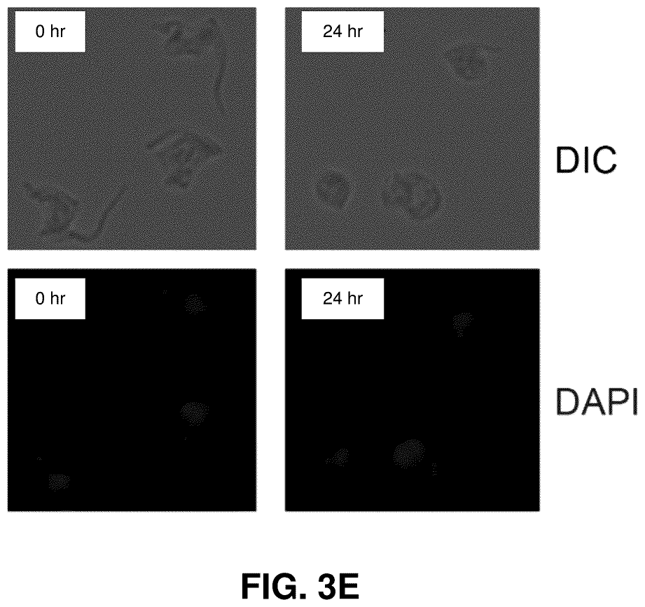

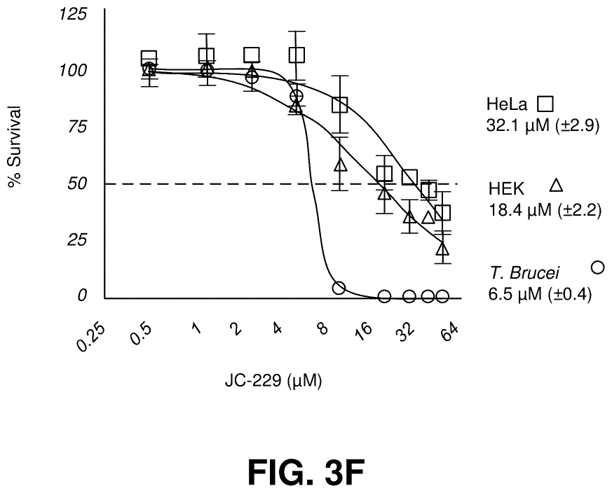

Without wishing to be bound to any particular theory, if JC-229 indeed binds and inhibits the functions of TbRPA1, as predicted from the structural model, JC-229-treated trypanosome cells should show similar phenotypes to TbRPA1-depleted cells without affecting TbRPA1 protein levels. Wild-type T. brucei cells was treated with 10 μM JC-229 for 48 hours and the cell-cycle profile and efficiency of DNA synthesis was monitored. TbRPA1, γH2A, VSG3, and Tubulin protein levels were also monitored (FIG. 3A, FIG. 3B, FIG. 3C and FIG. 3D).

Abnormalities in cell-cycle progression were observed at 8 and 16 hours after JC-229 treatment with a reduction in S phase population (FIG. 3A). In particular, the early S phase population (indicated with arrowheads in FIG. 3A) was diminished at the 16 and 24-hour time points. This result could be due to the role of RPA during the initiation of DNA replication. Replication origins are believed to not be activated in the absence of TbRPA1. As a consequence, cells are unable to enter S phase, thus reducing the early S phase population. At the 32-hour time point, a drastic increase in the sub-G1 population was observed (˜45%) and almost all cells (˜97%) lost the bulk of their DNA by 48 hours. JC-229 completely blocked new DNA synthesis after 8 hours of treatment (FIG. 3B). Importantly, JC-229 treatment did not change TbRPA1 protein levels (FIG. 3C and FIG. 3D), indicating that phenotypes observed in JC-229 treated cells occur due to the loss of TbRPA1 functions, such as ssDNA-binding activity, rather than the loss of protein itself. An approximately 5 and 7-fold increase in the levels of γH2A was observed after 16 and 24 hours of treatment respectively, and ˜3-fold increase in VSG3 protein levels at 24 hours. These data suggest that JC-229 likely targets an essential function(s) of the TbRPA1 protein. Microscopic analysis confirmed that JC-229 treatment leads to cell death (FIG. 3E). Trypanosome cells started losing their normal morphology (an elongated shape with flagellum attached to the body) after 16 hours of JC-229 treatment. After 24 hours, the majority of cells were dying, showing a rounded morphology that is typical of dying T. brucei cells (FIG. 3E). After 32 and 48 hours, unusually small cells that had <2C DNA were observed frequently.

JC-229 is an analog of compounds that inhibit the interaction between the recombinant DBD-F of HsRPA1 and ATRIP-derived peptides. Although JC-229 is not the same as the reported inhibitor, it is possible that JC-229 could still inhibit the functions of HsRPA1 DBD-F and/or DBD-AB, resulting in growth inhibition of human cells. Therefore, the selectivity of JC-229 toward T. brucei was examined by looking at its inhibitory effects on the growth of two human cell lines (HeLa and HEK293 cells). The toxicity of JC-229 was determined using the ALAMARBLUE® assay and found that JC-229 kills T. brucei cells more effectively than human cells (FIG. 3F). JC-229 exhibited EC₅₀ values of 6.5 μM (±0.4), 18.4 μM (±2.2), and 32.1 μM (±2.9) for T. brucei, HEK293, and HeLa, respectively: 100% inhibition of HEK293 and HeLa was not observed at 50 μM, the maximum concentration of JC-229. In addition, HEK293 and HeLa cells are barely inhibited (e.g. less than 20%) by JC-229 at 6.5 μM (EC₅₀ to T. brucei).

JC-229 Specifically Inhibits the ssDNA-Binding Activity of T. brucei RPA In Vitro

Measuring binding kinetics between the purified RPA protein and isotope or fluorescent dye-labeled oligonucleotides is a well-established method for studying RPA biochemical functions. To confirm the specific inhibition of TbRPA1 by JC-229 in vitro, electrophoretic mobility shift assays (EMSA) and microscale thermophoresis (MST) assays were performed using RPA proteins purified from E. coli and a 32-nucleotide ssDNA substrate (oligo dT₃₂) labeled with IRDye800 or Cy5 at the 5′ end.

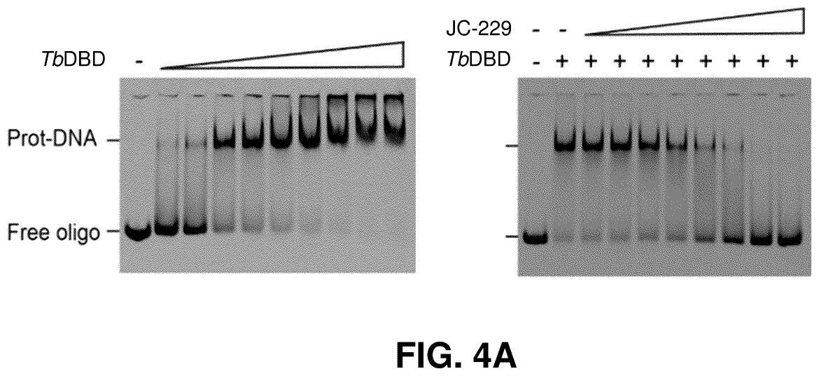

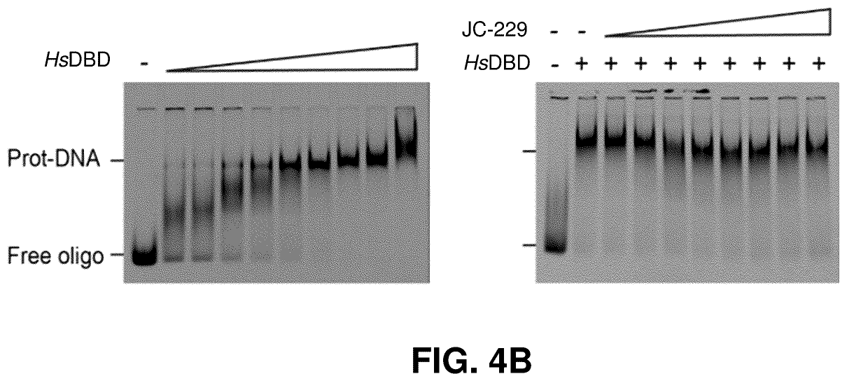

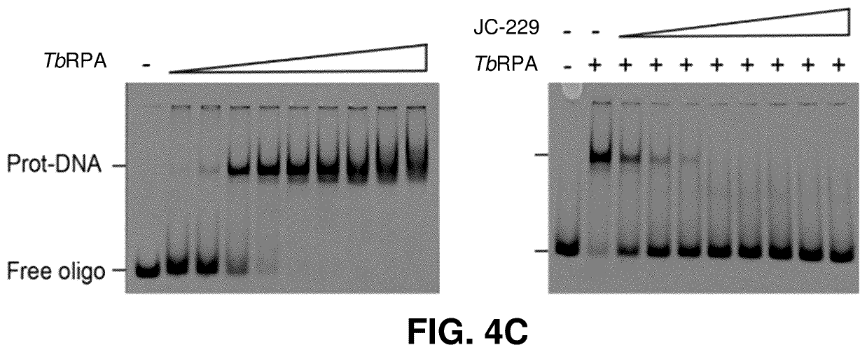

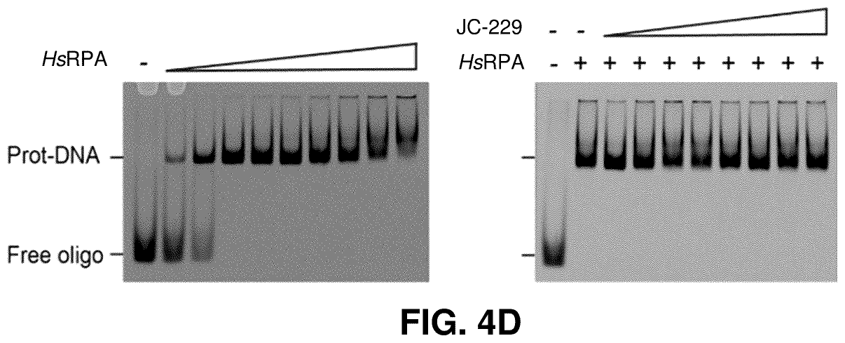

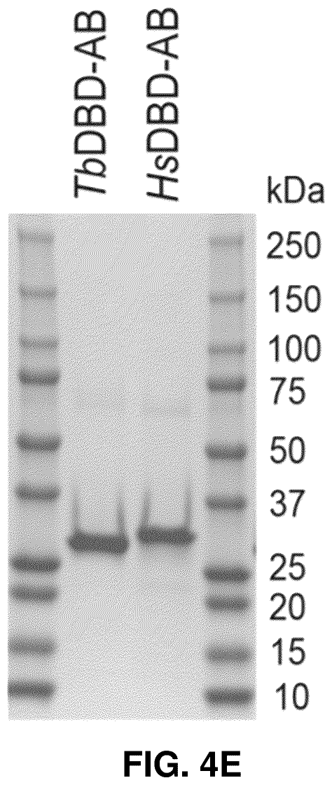

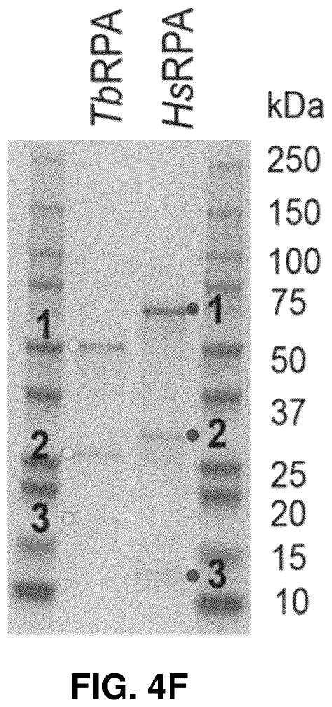

It is known that RPA subunits are not soluble by themselves, but they fold properly and form a stable complex of RPA when expressed together. Soluble TbRPA1 protein could not be obtained nor could the subunits of individual TbRPA2 or TbRPA3 be obtained in soluble form. Deleting DBD-C can increase the solubility of truncated HsRPA1 protein since this domain engages in complex formation by interacting with RPA2. Without RPA2, the DBD-C may misfold, making full length RPA1 insoluble. Instead, soluble 6xHis-tagged TbRPA1 DBD-AB (lacks DBD-C) were expressed from E. coli. TbRPA1 DBD-AB protein (in short, TbDBD-AB) was purified using Ni⁺⁺-nitrilotriacetic acid (Ni-NTA) purification (FIG. 4E). The purified TbRPA1 DBD-AB protein (amino acids 1-257) was used to study the ssDNA-binding properties of TbRPA1 and the inhibitory effect of JC-229.

Increasing concentrations of TbRPA1 DBD-AB proteins (0.05˜100 nM) were incubated with 5′IRDye800-labeled dT₃₂ probe. Unbound and protein-bound probes (RPA-ssDNA complex) were separated in a native PAGE gel and visualized with the LI-COR imaging system. Signals from the ssDNA-protein complex become stronger with increasing protein concentrations (FIG. 4A, left). To see if JC-229 inhibits the ssDNA-binding activity of TbRPA1 DBD-AB, TbRPA1 DBD-AB protein was pre-incubated with 2-fold serial dilutions of JC-229 (0.625˜80 μM) and then added 5′IRDye800-labeled oligos. The ssDNA binding of TbRPA1 DBD-AB was inhibited by JC-229 in a dose-dependent manner (FIG. 4A, right). The same assay was then performed using purified HsRPA1 DBD-AB (FIG. 4E), to see whether JC-229 specifically inhibits the T. brucei protein. Recombinant HsRPA1 DBD-AB protein bound ssDNA in a concentration-dependent manner (FIG. 4B, left). However, even the highest concentration of JC-229 (80 μM) did not inhibit ssDNA-binding activity of the human RPA1 protein (FIG. 4B, right). These data demonstrate that JC-229 targets TbRPA1 protein and, importantly, it exhibits specificity toward TbRPA1.

The RPA domains function as a complex, rather than as an individual subunit. The RPA complex has six DBDs with varying levels of activities, and all DBDs of RPA complex have a similar OB-fold, while the major ssDNA binding activity is present in DBD-A and DBD-B. To examine the ssDNA-binding properties in this more natural context, an EMSA was performed using recombinant RPA complexes. A vector was engineered for the simultaneous expression of 6xHis-tagged RPA1, RPA2, and RPA3 subunits in E. coli. Simultaneous expression of all three subunits in the same cell facilitates protein folding and complex formation. A full RPA complex can be purified by precipitating the 6xHis-tagged RPA1 subunit with Ni-NTA resin. Both TbRPA and HsRPA complexes were purified (FIG. 4F) and used in EMSA assays as above. The formation of ssDNA-protein complexes was dependent on the protein concentration for both TbRPA and HsRPA (FIG. 4C and FIG. 4D, gel images on the left). JC-229 inhibited TbRPA complex binding to ssDNA (FIG. 4C, right), with an even stronger effect than that seen in experiments with TbRPA1 DBD-AB. JC-229 had no effect on binding of the HsRPA complex to ssDNA (FIG. 4D, right). The data confirm the specificity of JC-229 against T. brucei RPA. All EMSA assays were performed in triplicate and signals were quantified and plotted (see U.S. Patent application 63/608,404).

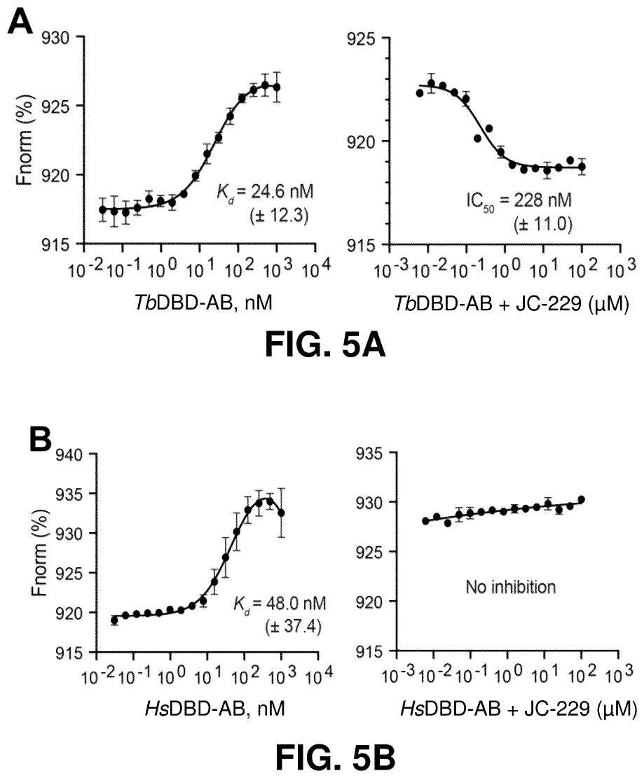

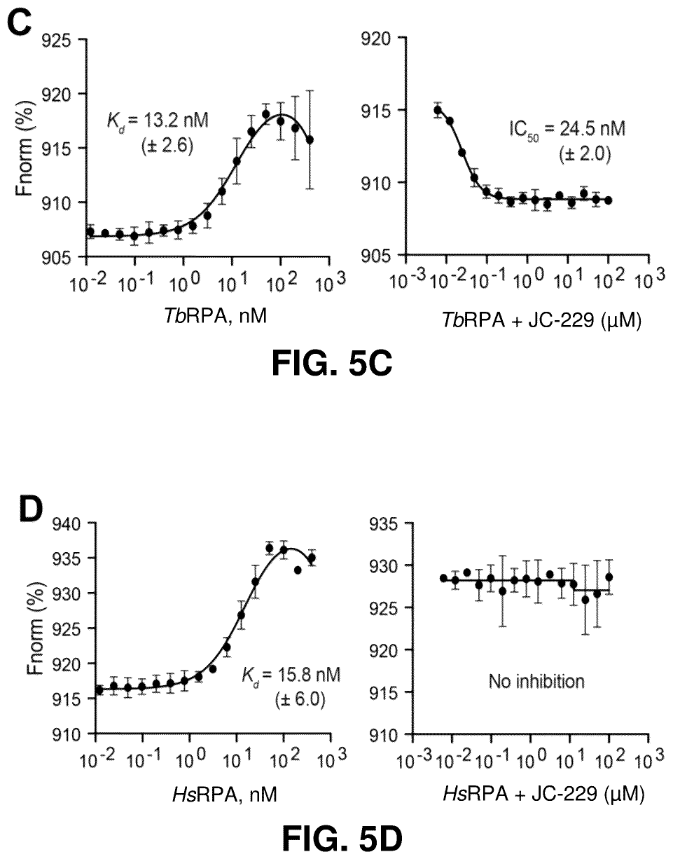

To accurately measure the binding constant and IC50 values, MST assays were performed using the 5′Cy5 labeled-dT₃₂ ssDNA probe and the proteins in the EMSA experiments above were used. Essentially the same trend was observed in binding and inhibition as in the EMSA experiments (FIGS. 5A-5D). The following K_{d} values were obtained for ssDNA binding: 24.6 nM for TbRPA1 DBD-AB, 13.2 nM for TbRPA complex, 48.0 nM for HsRPA1 DBD-AB, and 15.8 nM for HsRPA complex. JC-229 strongly inhibited the binding activity of TbRPA1 DBD-AB and the TbRPA complex, exhibiting IC₅₀ values of 228 nM and 24.5 nM (FIG. 5A, FIG. 5C: graphs on the right), respectively, while having no inhibitory effect on human RPA proteins (FIG. 5B, FIG. 5D: graphs on the right). TbRPA DBD-AB has a weaker binding affinity to ssDNA compared to the TbRPA complex (˜2 fold), but a much higher concentration of JC-229 is needed to inhibit the activity of TbDBD-AB than TbRPA complex (228 nM vs. 24.5 nM). Approximately three DBD-AB molecules can bind the 32 nt oligo probe, while only one RPA complex can sit on the same oligo since other DBDs (DBD-C, D & E) in the RPA complex also engage in ssDNA binding. Therefore, a higher concentration of JC-229 is required to inhibit the interaction of TbDBD-AB with oligo dT₃₂. The 3D structure of DBD-AB fragment might be slightly altered in the context of the natural RPA complex, allowing better binding to oligo dT₃₂.

E. coli has its own Single-Stranded DNA-Binding protein, SSB, the bacterial counterpart of RPA. To ensure that the purified proteins do not have residual E. coli SSB protein contamination, which could obscure the binding kinetics, two mutant TbRPA1 DBD-AB proteins were purified, one carrying a F64A mutation in DBD-A (F269 in human) and the other with a W188A mutation in DBD-B (W361 in human). Studies from other eukaryotes have shown that these are highly conserved aromatic residues that are essential for RPA-ssDNA interactions. The mutant proteins were tested for ssDNA binding using EMSA and MST. The F64A or W188A mutant proteins did not show ssDNA-binding activity, confirming that the binding activities observed in the experiments did not come from bacterial proteins.

T. brucei RPA1 DBD-A has the Binding Pocket for JC-229: Ser 105 in DBD-A is Important

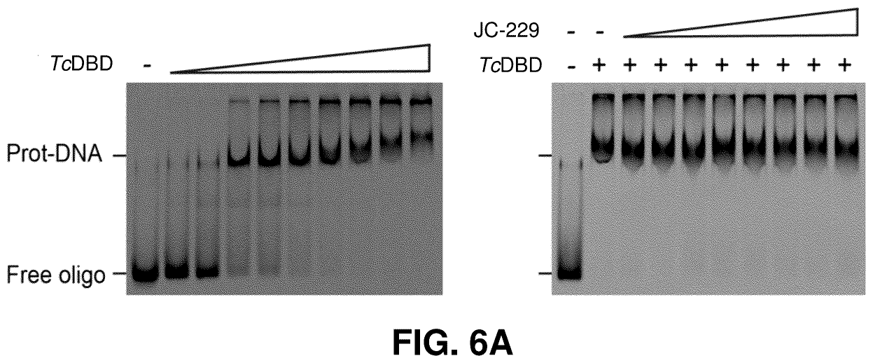

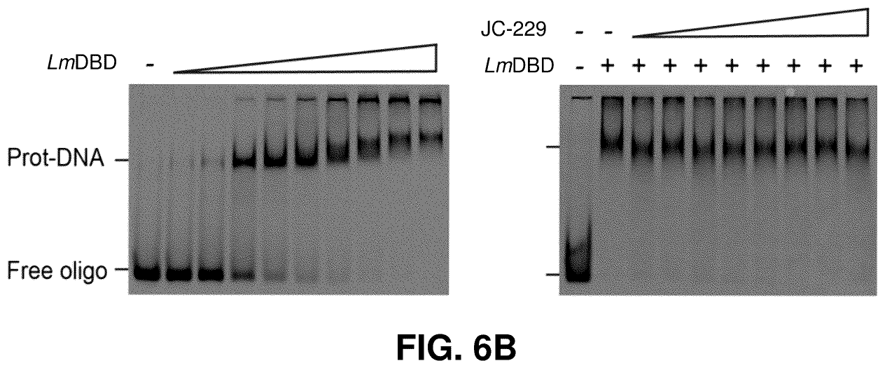

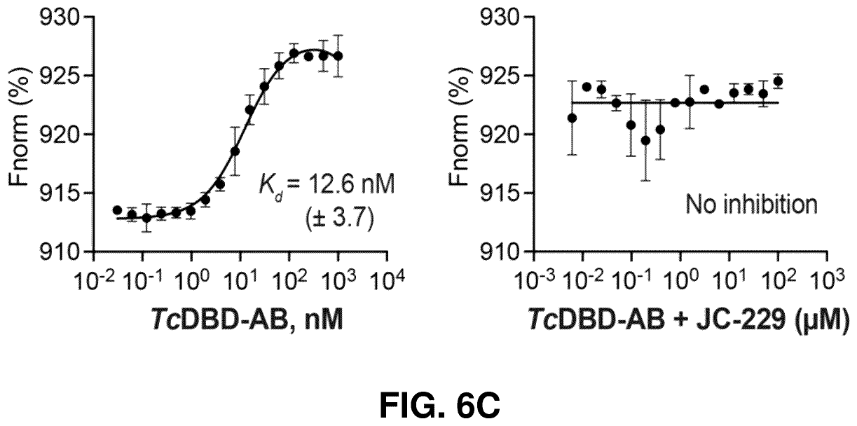

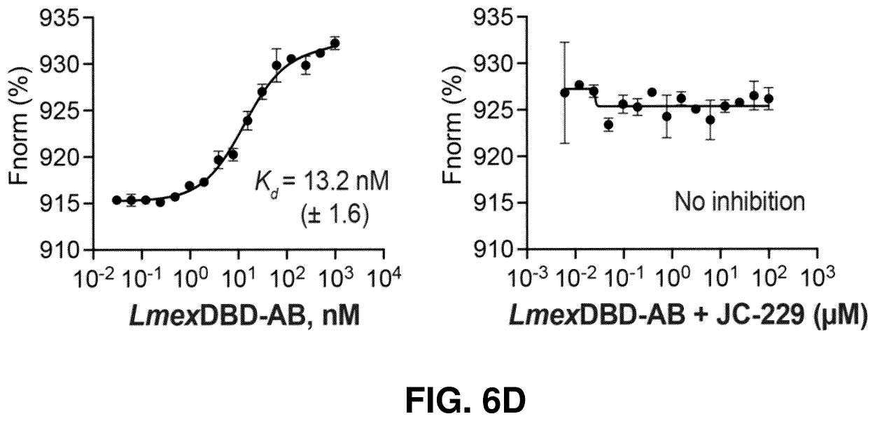

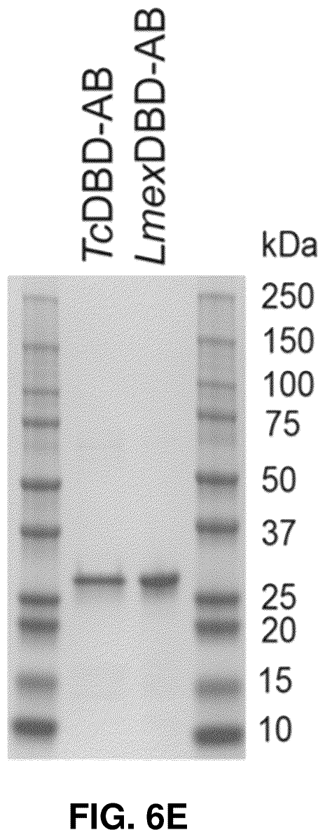

Sequence identity between DBD-ABs of T. brucei and T. cruzi is 83% and 72% between T. brucei and Leishmania. This high level of identity suggested that JC-229 may also inhibit the ssDNA-binding activity of T. cruzi and Leishmania RPA1. EMSA and MST experiments using recombinant TcRPA1 DBD-AB and L. mexicana RPA1 DBD-AB proteins tagged with 6xHis at the N-terminus. The DBD-AB of both TcRPA1 and LmexRPA1 showed strong ssDNA-binding activities (FIG. 6A, FIG. 6B, FIG. 6C and FIG. 6D; images and graphs on the left). FIG. 6E shows an SDS-PAGE gel showing purified fractions used for EMSA and MST assays. However, surprisingly, JC-229 did not inhibit either RPA1 (FIGS. 6A-D; images and graphs on the right). Tc and LmexRPA1 DBD-AB possess slightly higher ssDNA-binding affinities than TbRPA1 DBD-AB, as K_{d} values for Tc, Lmex, and Tb proteins are 13.2 nM, 12.6 nM, and 24.6 nM, respectively (FIGS. 6C & 6D, FIG. 5A). Signals from ssDNA-protein complexes in EMSA gels were quantified and plotted (see U.S. Patent application 63/608,404).

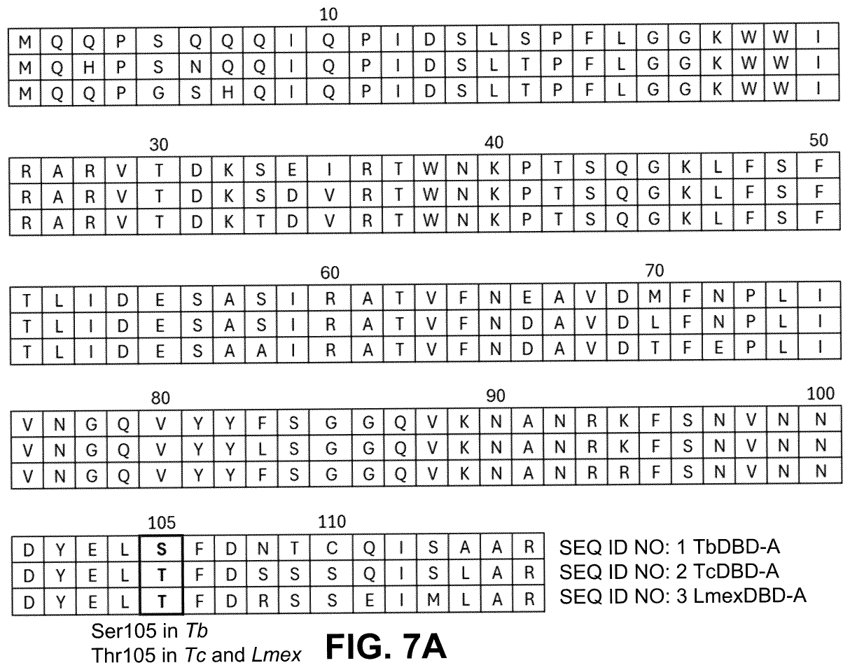

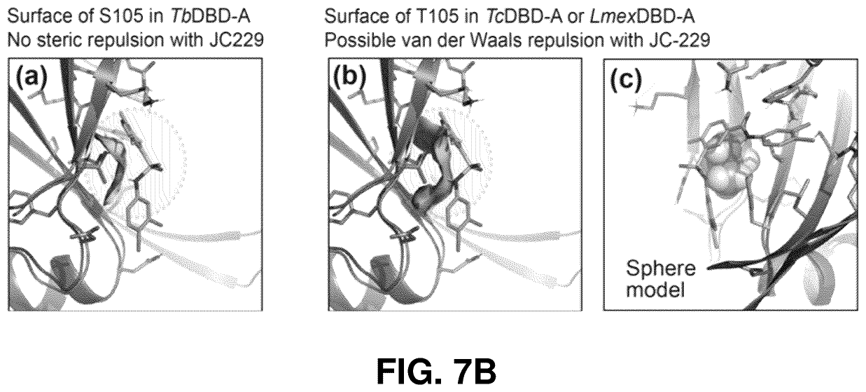

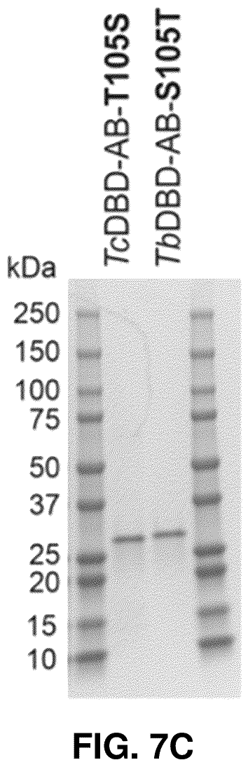

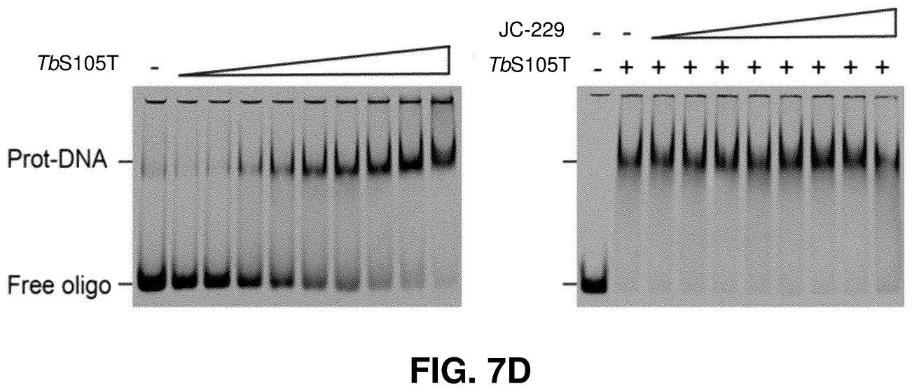

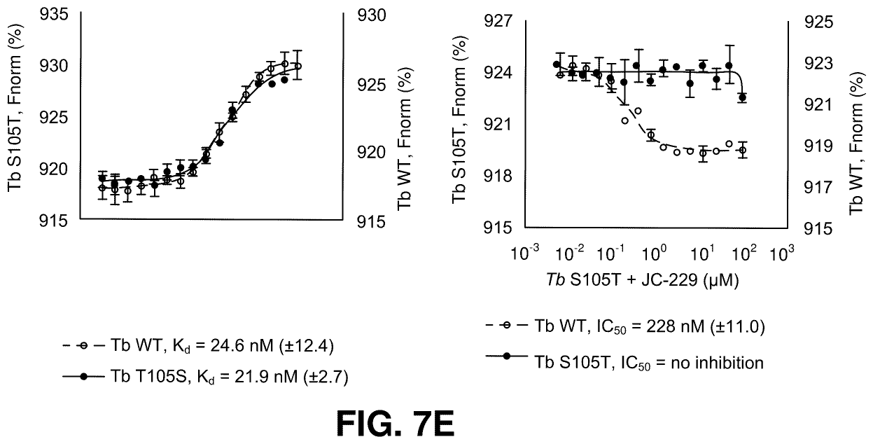

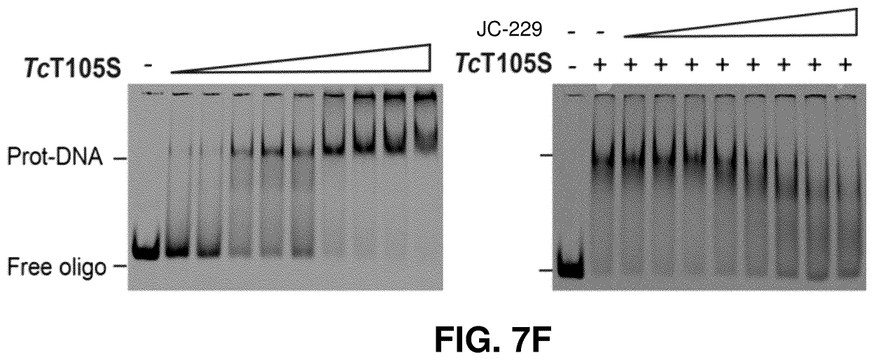

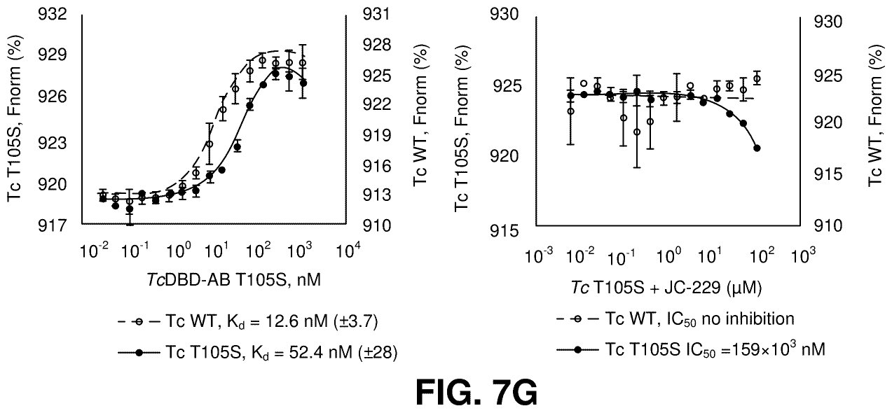

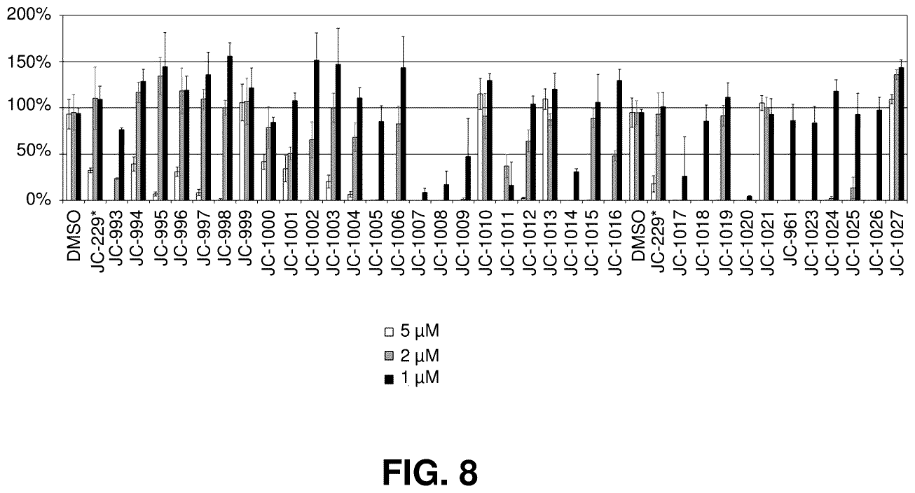

To explain the differential effects of JC-229 on these highly conserved proteins, we compared the potential binding sites of JC-229 in the DBD-A domains of Tb, Tc, and LmexRPA1 (FIG. 7A, FIG. 7B). Homology models of TcRPA1 and LmexRPA1 were generated. From the structure alignment of those models with JC-229-bound TbRPA1 structure, the amino acid at the 105-position is predicted to be important for differential effects: S105 of TbRPA1 vs. T105 of TcRPA1 and LmexRPA1 (FIG. 7B). No steric repulsion between JC-229 and the surface of S105 in TbDBD-A is expected based on the electrostatic potential surface of S105 in the docking model (FIG. 7B panel (a)). However, the methyl group of T105 of Tc or LmexDBD-A could cause van der Waals repulsion with JC-229 in the aligned structures (FIG. 7B panels (b), (c)). FIG. 7C depicts an SDS-PAGE gel showing purified fractions of TbRPA1 DBD-AB S105T and TcRPA1 DBD-AB T105S mutant proteins.

To validate the rationale for no inhibition to Tc or LmexRPA1, TbRPA1 DBD-AB-S105T and TcRPA1 DBD-AB-T105S mutant proteins were purified and analyzed using EMSA and MST (FIG. 7D, FIG. 7E, FIG. 7F, and FIG. 7G). While T. brucei S105T mutant protein showed a WT level of ssDNA-binding activity with a K_{d} of 21.9 nM (vs. 24.6 nM in WT), JC-229 does not inhibit its ssDNA-binding activity (FIG. 7D, 7E). This result agrees well with the docking models (FIG. 7B), confirming that the binding pocket of JC-229 is located in the DBD-A of TbRPA1 around S105, although its hydroxyl group is not directly involved in the interaction with JC-229. The ssDNA-binding activity of TcRPA1 DBD-AB T105S mutant protein was slightly decreased compared to WT (K_{d}=52.4 nM for mutant vs. 12.6 nM for WT), and it was inhibited by JC-229 at high concentrations (FIG. 7F, FIG. 7G). However, the inhibition against T. cruzi T105S mutant protein was not as robust as the effect of JC-229 on wild-type TbRPA1 DBD-AB protein. Collectively, the data confirm that JC-229 is a selective TbRPA1 inhibitor that blocks the binding of ssDNA to the DBD-A domain.

### DISCUSSION

The disclosed study is based upon functional and structural features of RPA1. T. brucei RPA1 is highly conserved and has an overall sequence identity of 34% with human RPA1, but it lacks the N-terminal domain which mediates protein-protein interactions in human RPA1. The functions of N-terminal DBD-F appear to have evolved from ssDNA binding to protein binding, as all DBDs including the DBD-F have the basic OB fold structure, a known motif found in many DNA-binding proteins. The computational models also showed that the binding pose of ATRIP-HsDBD-F resembles that of ssDNA-TbDBD-A. An ATRIP peptide binds the human DBD-F and DBD-AB proteins with similar affinities. T. brucei RPA is essential for survival, as is the case in other organisms. TbRPA2 and TbRPA3 are essential using RNAi depletion. Existing human RPA1 inhibitors, or their analogs, might also bind TbRPA1, probably with different affinities (likely lower), and that those inhibitors then can be tailored to target T. brucei RPA1 for selective killing of the parasite. Here, human RPA1 PPI inhibitors are repurposed. The disclosure also demonstrates that JC-229 selectively inhibits the ssDNA-binding function of TbRPA and the growth of T. brucei cells.

The model structure of TbRPA1 bound to JC-229 agrees well with the experimental results and could be used as a basis for further inhibitor design. In particular, the TbRPA1-S105T mutant retains its affinity for ssDNA but, as proposed by the structure, eliminates JC-229-mediated inhibition of ssDNA interaction. Since TcRPA1 and LmexRPA1 possess T105 at the equivalent position and JC-229 does not inhibit those proteins, the methyl unit of T105 seems to completely block JC-229 binding to the ssDNA-binding pocket of these RPA1 proteins. The electrostatic potential surface of T105 in the model structures overlaps with JC-229 in the ssDNA-binding pocket. This steric hinderance from T105 may prevent JC-229 interaction. Interestingly, the TcRPA1-T105S mutant is not as sensitive as WT TbRPA1 to JC-229. In fact, inhibition of the TcRPA1-T105S mutant is minimal even at the highest concentration of JC-229, compared to the WT TbRPA1. The IC₅₀ for TcRPA1-T105S was 159×10³ nM vs. 228 nM for WT-TbRPA1. As in other eukaryotes, the major ssDNA-binding activity is present in the DBD-A of TcRPA1. Therefore, although the structural alignment of all models indicates that all identical residues except S/T105 are oriented to the ssDNA binding pockets of Tb, Tc, and LmexRPA1 DBD-A, there might be some variations between the computer-generated models and the actual structures, and residues other than T105 may affect the ssDNA binding of TcRPA1 DBD-A.

Although TbRPA1 does not have the N-terminal domain seen in the human protein, TbRPA1 relocates to sites of DNA damage in response to DSB or replication stress. The DDR response, mediated by the ATR-RPA complex, seems to be conserved in T. brucei, but T. brucei ATRIP has not been identified yet. If RPA1 interaction with ATRIP is required for DDR activation in T. brucei, it is possible that JC-229 or its derivatives can also inhibit the interaction between TbRPA1 and a putative ATRIP ortholog. Other DBDs may fulfill the PPI function for the RPA complex in T. brucei. The DBD-A is the main ssDNA-binding domain in humans, yeasts, and T. cruzi, but it is not clear whether this is the case in T. brucei. There is no knowledge about molecular functions of TbRPA in the DDR in T. brucei. Additional biochemical characterization of TbRPA subunits and DBDs, and the identification of essential DDR factors including ATRIP, are necessary to understand the mechanisms of T. brucei RPA-mediated damage response. In addition, development of assay tools for complete functional assessment of TbRPA will provide better understanding of JC-229-mediated inhibition mechanisms.

In humans and yeasts, ATM/ATR (TEL1/MEC1 in yeasts) phosphorylates many DDR proteins, including RPA1 and RPA2, on their SQ/TQ motifs (Serine or Threonine residue followed by Glutamine). RPA1-T180 in human (S178 in budding yeast) is phosphorylated by ATR/ATM (MEC1/TEL1 in budding yeast). The T180 residue (or S178) is located at the border of the DBD-F and DBD-A. RPA1 and RPA2 are phosphorylated during S phase and levels of phosphorylation increase further in response to DNA damage and replication stress in humans and yeasts. Using cryo-EM and FRET (Fluorescence Resonance Energy Transfer) studies in the budding yeast, RPA1-S178 phosphorylation has been shown to be important for cooperative assembly of RPA complex on ssDNA. Unmodified RPA binds different lengths of ssDNA probes with similar affinities, while the S178D mutant (phosphomimetic substitution) binds the longer ssDNA better. The S178D mutant RPA binds an oligo dT₁₀₀ (approximately 3-4 RPA molecules can bind) with a lower affinity than unmodified RPA and RPA-ssDNA complexes are more flexible with S178D mutant. These observations indicate that phosphorylation affects the ssDNA-binding properties of RPA. In T. brucei RPA1, there are five SQ/TQ motifs, two are in the DBD-A (S5, S43) and three in DBD-C(T306, T341, S355). S43 and T341 and S355 are conserved in T. brucei, T. cruzi, and Leishmania. Because the in-vitro assays were performed with recombinant proteins purified from E. coli, all RPA proteins are unmodified.

### Methods

Trypanosoma brucei and Human Cell Lines

Trypanosoma brucei cells were cultured in HMI-9 medium supplemented with 10% fetal bovine serum (Hyclone) at 37° C. with 5% CO₂, as described in the scientific literature. Necessary antibiotics (InvivoGen) were added at the following concentrations; G418 at 2.5 g/ml, puromycin at 0.1 g/ml, phleomycin at 1 μg/ml, and hygromycin at 5 μg/ml. Cell lines and plasmids used in this study are listed in U.S. Patent application 63/608,404.

TbRPA1 (Tb927.11.9130) RNAi KD cell lines were generated in the 2T1 background that expresses a T7 RNA polymerase and a Tet repressor (TetR). The PHLEO marker in the 2T1 was replaced with NEO, generating the AMT2 strain (WT). AMT2 cells were transfected with a double stranded RNA inducible vector (pOH10 with p2T7TA backbone) targeting the TbRPA1 transcript. Two clones were obtained, AMT5 and AMT6 (denoted as KD1 and KD2).

Human cell lines, HeLa and HEK293 were maintained in Dulbecco's modified MEM (D-MEM, GIBCO) supplemented with 10% fetal bovine serum (GIBCO) at 37° C. with 5% CO₂.

### Construction of Plasmids for Induction of Recombinant Protein(s)

Plasmids for the expression of Tb, Tc, Hs, Lmex RPA1 DBD-AB from E. coli: pSR5 (T. brucei RPA1 DBD-AB), pSR13 (DBD-AB of T. cruzi RPA1, TCDM_07088), pSR15 (HsRPA1 DBD-AB), and pSR16 (DBD-AB of L. mexicana RPA1, LmxM.28.1820) were generated by PCR-amplifying the DBD-AB domain and cloning into a pET28a vector.

Plasmids for the simultaneous expression of three subunits of TbRPA or HsRPA complex in E. coli: To purify RPA complex from E. coli, a vector that expresses all three subunits of the TbRPA or HsRPA complex was generated, with only one subunit, RPA1, tagged with 6xHis and Thrombin cleavage site; pSR8 for TbRPA and pSR18i for HsRPA complex. Transcription of three subunits is driven individually by a T7 promoter. To insert all subunits in pET28a vector, TbRPA3 CDS (Tb927.9.11940) was amplified with a forward oligo containing HindIII-T7-LacO-RBS sequences and a reverse oligo containing a XhoI site. A HindIII/XhoI-digested fragment was ligated with a HindIII/XhoI-digested pET28a vector fragment, generating plasmid pSR6. Similarly, we PCR-amplified the TbRPA2 CDS (Tb927.5.1700) with a forward oligo containing BamHI-T7-LacO-RBS sequences and a reverse oligo containing a HindIII site, then digested with BamHI and HindIII, ligated with a BamHI/HindIII-digested pSR6 vector fragment, generating plasmid pSR7 (TbRPA2 & TbRPA3 in pET28a). The TbRPA1 CDS was inserted in pSR7 using NheI/BamHI digestion and ligation, generating the final plasmid pSR8 (6xHis-TbRPA1, TbRPA2 and TbRPA3 in pET28a).

To generate a vector containing human RPA subunits, 6xHis-RPA1, RPA2 and RPA3, as in pSR8, we used site-directed mutagenesis using two pairs of oligos and a template, p11d-tRPA(123) (AddGene). The upstream region (392 nt) of HsRPA1 was amplified with a forward oligo (HK-1256) and a reverse oligo tailed with 6xHis-Thrombin cleavage site sequences (HK-1232) and a 982 bp region of HsRPA1 CDS with a forward oligo tailed with 6xHis-Thrombin cleavage site (HK-1233) and a reverse oligo (HK-1257). Two PCR products were pooled at equimolar ratio and amplified with HK-1256 and HK-1257 oligos, which results in the insertion of 6xHis-Thrombin cleavage sequences at the N-terminus of HsRPA1 (pSR18i).

Plasmids for the expression of DBD-AB mutant proteins from E. coli, TbRPA1 DBD-AB with F64A or W188A or S105T and TcRPA1 DBD-AB with T105S: Site-directed mutagenesis (Quick change kit, NEB) was performed to insert an indicated mutation to TbDBD-AB in plasmid pSR5 (to introduce F64A, W188A, or S105T mutation) or to TcDBD-AB in plasmid pSR13 (to introduce T105S mutation).

Plasmids and oligonucleotides used in this study are listed in U.S. Patent application 63/608,404.

### Cell Growth and Viability Assay

To determine cell growth rate, WT and two TbRPA1 RNAi KD strains (KD1 and KD2) were seeded at a density of 1×10⁵ cells/ml and cultured with or without tetracycline for the induction of RNAi. Cells were counted every 12 hours for 2 days. To determine the viability of TbRPA1-depleted cells, ALAMARBLUE® assay (Thermo Fisher, an established colorimetric technique that detects metabolically active cells) was used. Cells from each time point were incubated with ALAMARBLUE® dye for 4-6 hours and percentage of live cells was determined by measuring resorufin converted from resazurin by reducing environment of live cells. Synergy H1 microplate reader (BioTek) was used with 560/590 nm (excitation/emission) filter settings and Relative Fluorescence Unit (RFU) values were obtained. The percentage of viable cells were calculated from the RFU values.

### Cell-Cycle Analysis and BrdU Pulse Experiments Using Flow Cytometry

About 10 million cells were collected after tetracycline induction or after JC-229 treatment. Cells were fixed with ice-cold 70% ethanol and incubated with 50 μg/ml PI (Sigma-Aldrich) and 200 μg/ml RNase A (Sigma-Aldrich) in 1×PBS (Corning) at 37° C. for 30 min in the dark. Samples were analyzed by flow cytometry (FACSVia, BD Biosciences) and FLOWJO®.

The bromo-2′ deoxyuridine (BrdU) incorporation assay was performed as described in the scientific literature. BrdU (Sigma-Aldrich) was added to cell cultures to a final concentration of 500 μM and incubated for 40 min at 37° C. About 20 million cells were harvested and fixed in ice-cold 70% ethanol. DNA in fixed cells was denatured in 0.1N HCl (Fisher Scientific) with 0.5% Tween 20 (Sigma-Aldrich) and 1% BSA (Jackson Immunoresearch) for 20 min at room temperature. To stain newly incorporated DNA, fixed cells were incubated with 1 μg/ml mouse anti-BrdU antibody (Fisher Scientific) at room temperature for 2.5 hours and then with 4 μg/ml donkey anti-mouse Alexa 488 (Invitrogen) at room temperature for 1 hour in the dark. Bulk DNA was stained with 5 μg/ml PI by incubating at room temperature for 30 min in the dark. Samples were analyzed by flow cytometry (FACSVia, BD Biosciences) and FLOWJO®.

### Microscopy

For microscopic analysis, untreated or JC-229 treated cells were seeded, and fixed with 1% paraformaldehyde for 10 min at room temperature. Cells were then washed with 1× PBS and stained with DAPI (0.5 μg/ml). Images were captured using fluorescence microscopy (EVOS M5000, Thermo Fisher Scientific) at 40× magnification and edited with ADOBE® Photoshop.

### Immunoblotting

Cells were harvested at indicated time points and suspended in Laemmli buffer (Bio-Rad) at 0.25 million cells per μl. Denatured proteins from whole cells were separated on a 4-20% gradient SDS-PAGE gel (BioRAD) and transferred to nitrocellulose membrane (GE Healthcare). The membranes were probed with polyclonal antibodies against TbRPA1 (a gift from Bibo Li laboratory) or γH2A (generated by ABclonal for this study), or monoclonal mouse antibodies against VSG3 (Antibody and Bioresource Core facility, MSKCC) or tubulin (Sigma) in 1× PBS with 0.1% Tween 20 and 1% milk (BioRAD) at 4° C. overnight. The membranes were then incubated with secondary antibodies conjugated with Horseradish Peroxidase (HRP) (GE healthcare) for the detection of corresponding proteins using chemiluminescence (Hyglo from Denville Scientific, ChemiDoc Imaging System from BioRAD).

Cell Viability of JC-229-Treated T. brucei and Human Cells

The initial assessment of JC-229's inhibitory effect on T. brucei cell growth was performed at 50 μM concentration. Trypanosome cells were seeded at 1×10³ cells/ml density in 180 μl in a 96-well culture plate in triplicate. These cells were incubated with 50 μM JC-229 for 24, 48, and 72 hours. DMSO was used as a vehicle control at 0.0075%. JC-229-treated culture and control cultures were analyzed with the ALAMARBLUE® dye as described above.

To obtain the EC₅₀ value of JC-229, T. brucei cells were seeded at 1×10³ cells/ml density in 180 μl in a 96-well culture plate in triplicate. These cells were incubated for 72 hours with increasing concentrations of JC-229, (0.5 μM to 50 μM) and cell viability was determined using the ALAMARBLUE® dye as described above.

To determine the inhibitory effect of JC-229 on human cells, HeLa and HEK293 cells were suspended in 180 μl D-MEM with 10% FBS and plated at a density of 2×10⁴ cells/ml in triplicate. Cells were adhered overnight, and the medium was replaced, and cells were then incubated for 72 hours with increasing concentrations of JC-229 (0.5 μM to 50 μM) and cell viability was determined using the ALAMARBLUE® dye as described above. Percent viability was plotted using GraphPad Prism software. Curves generated from human cell data did not fit the 4PL, as they did not reach the bottom plateau with the highest concentration of JC-229 used. The EC₅₀ values were estimated using the concentration of JC-229 that cause 50% reduction of cell viability (inhibitor vs. normalized response, variable slope).

### Synthesis of JC-229

3-(chlorosulfonyl)-4-methylbenzoic acid (reagent 1): Chlorosulfonic acid (6 ml, 90 mmol, 13 eq) was added to 4-methylbenzoic acid (1 g, 7.34 mmol) and the mixture was heated at 100° C. for 2 hours. The reaction was then cooled to room temperature and ice water was added to the mixture until white precipitates formed. The white precipitate was collected and dried under vacuum to give the product (1.44 g, 84% yield). ¹H NMR (400 MHz, DMSO-d6) δ 8.32 (d, J=1.9 Hz, 1H), 7.77 (dd, J=7.8 Hz, 2.0 Hz, 1H), 7.26 (d, J=7.9 Hz, 1H), 2.58 (s, 3H). The ¹H NMR spectroscopy data match with the report.

3-(chlorosulfonyl)-4-methylbenzoyl chloride (reagent 2): Thionyl chloride (12 ml, 169.5 mmol, 77 eq) was added to 3-(chlorosulfonyl)-4-methylbenzoic acid (1) (500 mg, 2.13 mmol), and the reaction mixture was heated at 75° C. for 12 hours. The thionyl chloride was distilled to produce the titled product as a red oil, which was used for the next reaction without further purification.

N-(3,4-dichlorophenyl)-3-(N-(3,4-dichlorophenyl)sulfamoyl)-4-methylbenzamide (JC-229): 3,4-dichloroaniline (691 mg, 4.27 mmol) was added to a solution of 3-(chlorosulfonyl)-4-methylbenzoyl chloride (180 mg, 0.71 mmol) in toluene (5 ml). The reaction mixture was heated at 75° C. for 12 hours. The reaction was then cooled to room temperature and diluted with ethyl acetate and was washed with saturated NaHCO₃, brine, and the organic layer was dried over MgSO4 and concentrated under reduced pressure. The crude mixture was purified using flash chromatography using hexane and ethyl acetate and further purified using preparative HPLC providing the desired product (JC-229) as an off-white solid (297 mg, 83% yield). ¹H NMR (400 MHz, DMSO-d₆) δ 11.04 (s, 1H), 10.69 (s, 1H), 8.51 (d, J=2.0 Hz, 1H), 8.14 (dd, J=6.9 Hz, 2.2 Hz, 2H), 7.76 (dd, J=8.9 Hz, 2.4 Hz, 1H), 7.62 (dd, J=14.2 Hz, 8.5 Hz, 2H), 7.50 (d, J=8.8 Hz, 1H), 7.28 (d, J=2.6 Hz, 1H), 7.08 (dd, J=8.8 Hz, 2.6 Hz, 1H), 2.66 (s, 3H). ¹³C NMR (101 MHz, DMSO-d₆) δ 163.97, 140.88, 138.91, 137.46, 137.31, 133.10, 132.28, 132.24, 131.51, 131.32, 130.85, 130.59, 128.88, 125.57, 125.41, 121.63, 120.41, 119.81, 118.45, 19.70. MS(ESI) calculated for C₂₂H₁₈C₁₄N₃O₃S [M⁺CH₃CN⁺H]⁺543.97, found 546.0.

### Structure Modeling of RPA1 Proteins and Computational Analysis of Induced Fit Model

The homology model of TbRPA1 was built by using a protein structure homology-modeling server (SWISS-MODEL). Template search showed that the 4gop.1.C structure (Ustilago maydis RPA1 and ssDNA complex) has 36% sequence identity with TbRPA1, and it was selected for building the homology model. The model structure was refined with Protein Preparation Wizard implemented in Maestro 12. The protein structure was imported into workspace and preprocessed to assign bond orders, add hydrogen atoms, create zero-order bonds to metals, create disulfide bonds, and to delete water molecules beyond 5 Å from hetero groups. In addition, missing atoms in residues and missing loops were added using Prime to generate a complete protein structure. The protein structure was further refined via automated H-bond assignment and restrained minimization with OPLS 2005 force field by converging heavy atoms to 0.3 Å RMSD. DBD-A and DBD-B domains of the refined TbRPA1 model structure were selected for Induced Fit docking of JC-229. Initially, the ligand structure (JC-229) was built using the 3D build module and prepared by the Ligprep module for molecular docking. Induced Fit docking was performed using the ‘standard protocol’ (SP) mode, and T62, F64, and K89 were selected to define the receptor grid. The size of inner grid box was set to 10 Å (default value), and its outer grid box was automatically determined, which includes the most part of the DBD-A structure. Ligand ring conformation was sampled with 2.5 kcal/mol energy window. Residues within 5 Å of the ligand in the initial docking were given flexibility during the prime refinement process, and the ligand was re-docked using the Glide SP mode. The Induced Fit Docking (IFD) score was calculated by the Prime energy, glide score, and glide Coulomb energy. The final binding pose was selected based on the IFD scores and the binding poses of Induced Fit docking models.

### Purification of Recombination Proteins

A protein expression plasmid was transformed into BL21 competent cells (Invitrogen) and selected in an appropriate antibiotic (ampicillin or kanamycin). Expression of a recombinant protein was induced with Isopropyl B-D-1-thiogalactopyranoside (IPTG) at 1 mM concentration. Induction was carried out for 3 hours at 37° C. for all RPA1 DBD-AB proteins and for 16 hours at 18° C. for TbRPA complex and HsRPA complex. 50 ml of IPTG-induced cells were harvested by centrifugation at 13,000 rpm at 4° C. and aliquoted (5 microcentrifuge tubes), snap-frozen, and store at −80° C. For purification, frozen cell pellets were resuspended and lysed in B-PER® Complete Bacterial Protein Extraction Reagent (Thermo Scientific) with 1 mM phenylmethylsulfonylfloride (PMSF) and EDTA-free Protease/phosphatase-inhibitor cocktail (Thermo Scientific) for 10 min with inverting the tubes a few times. The whole cell lysate was centrifuged at 13,000 rpm at 4° C. The supernatant diluted with equal volume of Equilibrium Buffer (2× PBS with 10 mM imidazole, pH 7.4) was mixed with Ni-NTA resin (washed twice with Equilibrium Buffer) and incubated for 2 hours at 4° C. on a tube rotator. Unbound fraction (flow through) was collected by centrifugation at 3,000 rpm for 1 min at 4° C. The resin was washed four times with Wash Buffer (2× PBS with 25 mM imidazole). 6xHis tagged proteins were eluted with Elution Buffer (2× PBS with 500 mM imidazole). Samples from each step were analyzed on a 4-20% SDS PAGE gel. Eluted fractions containing a high amount of recombinant protein was desalted by size-exclusion chromatography (Zeba Spin Desalting Column, Thermo Scientific). Purified proteins were snap-frozen and stored at −80° C. and used for assays below.

### Electrophoretic Mobility Shift Assays (EMSA)

ssDNA-binding assay: 5′IRDye800 labeled oligo dT₃₂ was purchased from Integrated DNA Technologies (IDT). Increasing concentrations of recombinant protein (from 0.05 nM to 100 nM) were incubated with 2 nM of 5′IRDye800-labeled oligo dT₃₂ in binding buffer (20 mM Tris, pH 7.5, 10 mM NaCl, 10 mM MgCl2, 2 mM dithiothreitol (DTT), 10% glycerol and 0.1 mg/ml BSA) for 15 min at room temperature. 4 μl of 6× Orange dye (Sigma) was added to the 20 μl reaction and from that 8 μl was separated on a non-denaturing polyacrylamide gel in 1× Tris-Borate-EDTA (TBE, Invitrogen) buffer; 4% gel for RPA complex and 8% for DBD-AB proteins. Gels were pre-run for 1 hour at a constant 100 V. Protein-ssDNA complex and unbound ssDNA were separated by running the gel at a constant 80 V at 4° C. in the dark, and then visualized using Li-COR Odyssey imaging system and quantified with ImageJ software.

Inhibition of DNA binding activity by JC-229: To determine the inhibitory effect of JC-229, increasing concentrations of JC-229 (from 2.5 μM to 320 M) were incubated with recombinant protein (50 nM for DBD-AB and 12.5 nM RPA) in 10 μl reaction volume for 15 min at room temperature. To each of the reaction mix, 10 μl of 5′IRDye800-labeled oligo dT₃₂ was added to a final concentration of 2 nM and the reaction was then incubated another 15 min at room temperature. Samples were separated on a non-denaturing polyacrylamide gel and analyzed as described above in ‘ssDNA-binding assay’.

### MicroScale Thermophoresis (MST)

MST experiments were performed on Monolith NT.115 instrument with red filter (Nanotemper Technologies) in MST binding buffer (20 mM Tris, pH 7.5, 10 mM NaCl, 10 mM MgCl₂, 2 mM DTT, 10% glycerol and 0.1 mg/ml BSA) using premium-coated capillaries (Nanotemper Technologies). MST experiments were performed with 40% LED power and 40% MST power at 22° C.

ssDNA-binding assay: The 5′Cy5 labeled-oligo dT32 was purchased from IDT. Increasing concentrations of purified protein (from 0.06 nM to 2,000 nM for DBD-AB proteins; from 0.02 nM to 800 nM for RPA complexes) were incubated with equal volume (10 μl) of 5′Cy5-labeld oligo dT₃₂ (10 nM final concentration) in MST binding buffer for 15 min at room temperature. Samples were then loaded into MST capillaries.

Inhibition of DNA binding activity by JC-229: Increasing concentrations of JC-229 (from 0.024 μM to 400 M) were incubated within a range of 15 nM to 30 nM recombinant protein in 10 μl volume for 15 min at room temperature. To these, 10 μl of 5′Cy5-labeled oligo dT₃₂ was added to a final concentration of 10 nM and incubated for another 15 min at room temperature. Samples were then loaded into MST capillaries. One site binding was used to obtain a dissociation constant (K_{d}) and standard 4-parameter logistic curve (Sigmoidal curve) analysis to obtain IC₅₀ value of JC-229.

### Statistical Information and Image Preparation

GraphPad Prism software (version 9.3.1) was used to prepare all graphs, calculate K_{d}, IC₅₀ and EC₅₀ values, and to perform statistical tests. FLOWJO® (v.10) was used to analyze flow cytometric data. Statistical significance was determined using student's t-test and final figures were prepared using Illustrator (ADOBE®).

Additional, non-limiting, examples of suitable inhibitors are provided below.

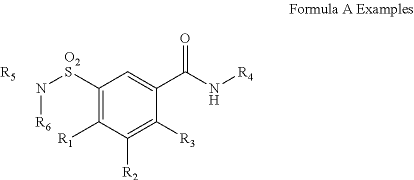

TABLE 1
Formula A examples

| R₅ | R₆ | R₁ | R₂ | R₃ | R₄ | ID |
|---|---|---|---|---|---|---|
| 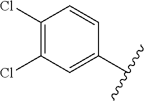 [SMILES: *c1ccc(Cl)c(Cl)c1] | H | CH₃ | H | H | 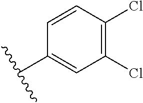 [SMILES: *c1ccc(Cl)c(Cl)c1] | JC-229 |
| 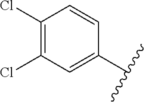 [SMILES: *c1ccc(Cl)c(Cl)c1] | H | CH₃ | H | H | 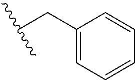 [SMILES: *Cc1ccccc1] | JC-856 |
| 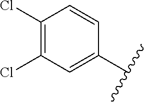 [SMILES: *c1ccc(Cl)c(Cl)c1] | H | CH₃ | H | H | 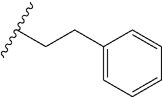 [SMILES: *CCc1ccccc1] | JC-859 |
| 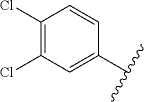 [SMILES: *c1ccc(Cl)c(Cl)c1] | H | CH₃ | H | H | 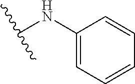 [SMILES: *Nc1ccccc1] | JC-860 |
| 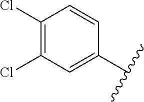 [SMILES: *c1ccc(Cl)c(Cl)c1] | H | CH₃ | H | H | 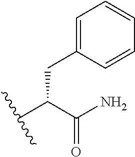 [SMILES: *[C@H](Cc1ccccc1)C(N)=O] | JC-861 |
| 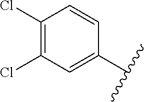 [SMILES: *c1ccc(Cl)c(Cl)c1] | H | CH₃ | H | H | 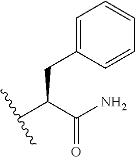 [SMILES: *[C@@H](Cc1ccccc1)C(N)=O] | JC-862 |
| 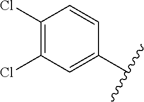 [SMILES: *c1ccc(Cl)c(Cl)c1] | H | CH₃ | H | H | 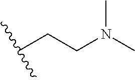 [SMILES: *CCN(C)C] | JC-863 (TFA salt) |
| 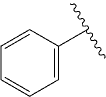 [SMILES: *c1ccccc1] | H | CH₃ | H | H | 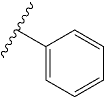 [SMILES: *c1ccccc1] | JC-625 |
| 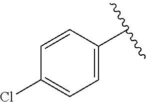 [SMILES: *c1ccc(Cl)cc1] | H | CH₃ | H | H | 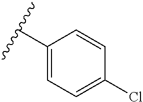 [SMILES: *c1ccc(Cl)cc1] | JC-626 |
| 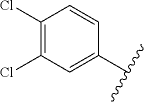 [SMILES: *c1ccc(Cl)c(Cl)c1] | H | H | H | H | 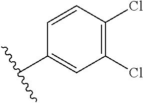 [SMILES: *c1ccc(Cl)c(Cl)c1] | JC-627 |
| 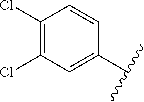 [SMILES: *c1ccc(Cl)c(Cl)c1] | H | CH₃ | H | H | 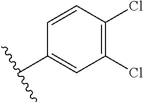 [SMILES: *c1ccc(Cl)c(Cl)c1] | JC-628 |
| 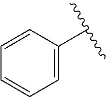 [SMILES: *c1ccccc1] | H | CH₃ | H | H | 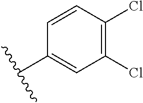 [SMILES: *c1ccc(Cl)c(Cl)c1] | JC-629 |
| 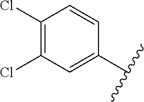 [SMILES: *c1ccc(Cl)c(Cl)c1] | H | CH₃ | H | H | 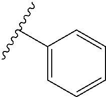 [SMILES: *c1ccccc1] | JC-630 |
| 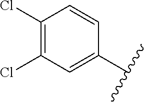 [SMILES: *c1ccc(Cl)c(Cl)c1] | H | CH₃ | H | H | CH₂CH₂OH | JC-864 |
| 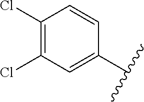 [SMILES: *c1ccc(Cl)c(Cl)c1] | H | CH₃ | H | H | 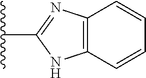 [SMILES: *c1nc2ccccc2[nH]1] | JC-865 |
| 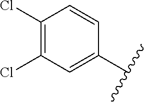 [SMILES: *c1ccc(Cl)c(Cl)c1] | H | CH₃ | H | H | 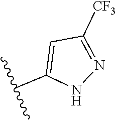 [SMILES: *c1cc(C(F)(F)F)n[nH]1] | JC-866 |
| 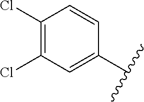 [SMILES: *c1ccc(Cl)c(Cl)c1] | H | H | H | CH₃ | 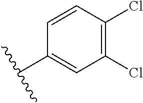 [SMILES: *c1ccc(Cl)c(Cl)c1] | JC-858 |
| 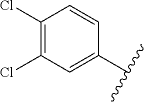 [SMILES: *c1ccc(Cl)c(Cl)c1] | H | Cl | H | H | 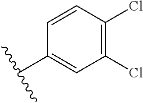 [SMILES: *c1ccc(Cl)c(Cl)c1] | JC-853 |
| 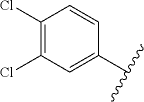 [SMILES: *c1ccc(Cl)c(Cl)c1] | H | Br | H | H | 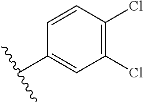 [SMILES: *c1ccc(Cl)c(Cl)c1] | JC-855 |
| 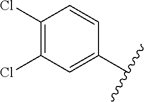 [SMILES: *c1ccc(Cl)c(Cl)c1] | H | OH | H | H | 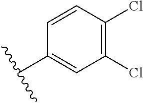 [SMILES: *c1ccc(Cl)c(Cl)c1] | JC-907 |
| 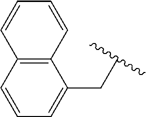 [SMILES: *Cc1cccc2ccccc12] | H | CH₃ | H | H | 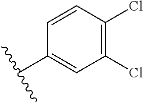 [SMILES: *c1ccc(Cl)c(Cl)c1] | JC-887 |
| 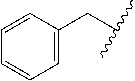 [SMILES: *Cc1ccccc1] | H | CH₃ | H | H | 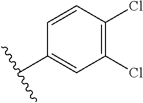 [SMILES: *c1ccc(Cl)c(Cl)c1] | JC-857 |
| 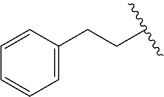 [SMILES: *CCc1ccccc1] | H | CH₃ | H | H | 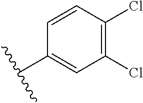 [SMILES: *c1ccc(Cl)c(Cl)c1] | JC-854 |
| Ph(CH₂)₃ | H | CH₃ | H | H | 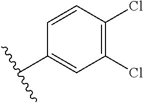 [SMILES: *c1ccc(Cl)c(Cl)c1] | JC-908 |
| 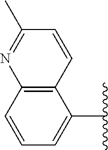 [SMILES: *c1cccc2nc(C)ccc12] | H | CH₃ | H | H | 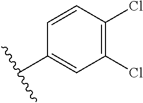 [SMILES: *c1ccc(Cl)c(Cl)c1] | JC-913 |
| 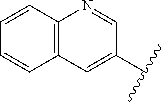 [SMILES: *c1cnc2ccccc2c1] | H | CH₃ | H | H | 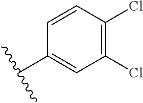 [SMILES: *c1ccc(Cl)c(Cl)c1] | JC-914 |
| 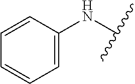 [SMILES: *Nc1ccccc1] | H | CH₃ | H | H | 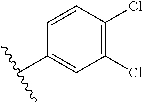 [SMILES: *c1ccc(Cl)c(Cl)c1] | JC-868 |
| 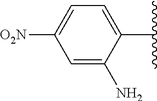 [SMILES: *c1ccc([N+](=O)[O-])cc1N] | H | CH₃ | H | H | 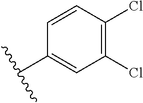 [SMILES: *c1ccc(Cl)c(Cl)c1] | JC-892 |
| 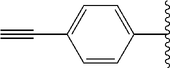 [SMILES: *c1ccc(C#C)cc1] | H | CH₃ | H | H | 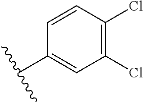 [SMILES: *c1ccc(Cl)c(Cl)c1] | JC-901 |
| 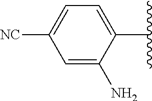 [SMILES: *c1ccc(C#N)cc1N] | H | CH₃ | H | H | 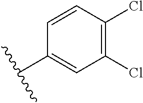 [SMILES: *c1ccc(Cl)c(Cl)c1] | JC-889 |
| 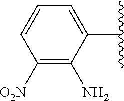 [SMILES: *c1cccc([N+](=O)[O-])c1N] | H | CH₃ | H | H | 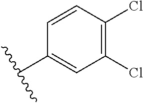 [SMILES: *c1ccc(Cl)c(Cl)c1] | JC-899 |
| 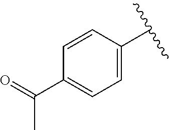 [SMILES: *c1ccc(C(C)=O)cc1] | H | CH₃ | H | H | 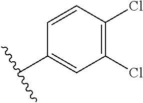 [SMILES: *c1ccc(Cl)c(Cl)c1] | JC-909 |
| 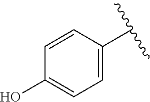 [SMILES: *c1ccc(O)cc1] | H | CH₃ | H | H | 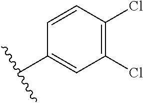 [SMILES: *c1ccc(Cl)c(Cl)c1] | JC-912 |
| 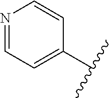 [SMILES: *c1ccncc1] | H | CH₃ | H | H | 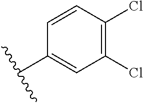 [SMILES: *c1ccc(Cl)c(Cl)c1] | JC-915 |
| 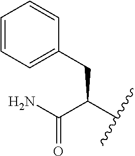 [SMILES: *[C@H](Cc1ccccc1)C(N)=O] | H | CH₃ | H | H | 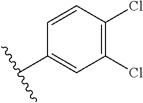 [SMILES: *c1ccc(Cl)c(Cl)c1] | JC-869 |
| 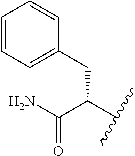 [SMILES: *[C@@H](Cc1ccccc1)C(N)=O] | H | CH₃ | H | H | 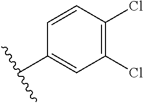 [SMILES: *c1ccc(Cl)c(Cl)c1] | JC-870 |
| (CH₃)₂N(CH₂)₂ | H | CH₃ | H | H | 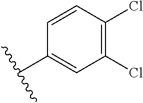 [SMILES: *c1ccc(Cl)c(Cl)c1] | JC-871 |
| HO(CH₂)₂ | H | CH₃ | H | H | 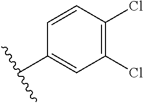 [SMILES: *c1ccc(Cl)c(Cl)c1] | JC-872 |
| 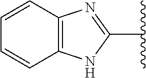 [SMILES: *c1nc2ccccc2[nH]1] | H | CH₃ | H | H | 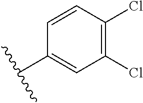 [SMILES: *c1ccc(Cl)c(Cl)c1] | JC-873 |
| 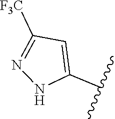 [SMILES: *c1cc(C(F)(F)F)n[nH]1] | H | CH₃ | H | H | 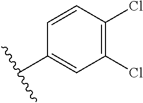 [SMILES: *c1ccc(Cl)c(Cl)c1] | JC-874 |
| 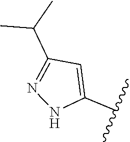 [SMILES: *c1cc(C(C)C)n[nH]1] | H | CH₃ | H | H | 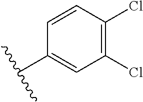 [SMILES: *c1ccc(Cl)c(Cl)c1] | JC-888 |
| 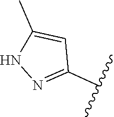 [SMILES: *c1cc(C)[nH]n1] | H | CH₃ | H | H | 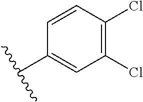 [SMILES: *c1ccc(Cl)c(Cl)c1] | JC-894 |
| 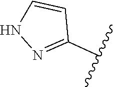 [SMILES: *c1cc[nH]n1] | H | CH₃ | H | H | 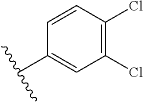 [SMILES: *c1ccc(Cl)c(Cl)c1] | JC-902 |
| 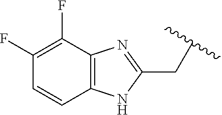 [SMILES: *Cc1nc2c(F)c(F)ccc2[nH]1] | H | CH₃ | H | H | 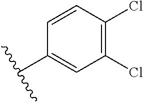 [SMILES: *c1ccc(Cl)c(Cl)c1] | JC-903 (TFA salt) |
| 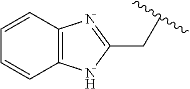 [SMILES: *Cc1nc2ccccc2[nH]1] | H | CH₃ | H | H | 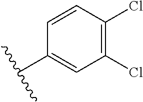 [SMILES: *c1ccc(Cl)c(Cl)c1] | JC-905 (TFA salt) |
| HO(CH₂)₂O(CH₂)₂ | H | CH₃ | H | H | 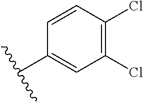 [SMILES: *c1ccc(Cl)c(Cl)c1] | JC-890 |
| HO(CH₂)₄ | Bz | CH₃ | H | H | 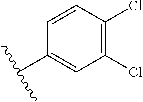 [SMILES: *c1ccc(Cl)c(Cl)c1] | JC-891 |
| (HOCH₂)₃C | H | CH₃ | H | H | 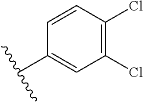 [SMILES: *c1ccc(Cl)c(Cl)c1] | JC-893 |
| HO(CH₂)₄ | H | CH₃ | H | H | 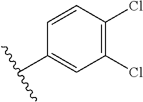 [SMILES: *c1ccc(Cl)c(Cl)c1] | JC-895 |
| HO(CH₂)₅ | H | CH₃ | H | H | 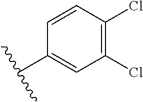 [SMILES: *c1ccc(Cl)c(Cl)c1] | JC-897 |
| HO(CH₂)₆ | H | CH₃ | H | H | 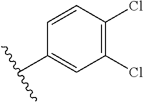 [SMILES: *c1ccc(Cl)c(Cl)c1] | JC-898 |
| Boc—(NH)—(CH₂)₂ | H | CH₃ | H | H | 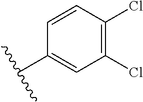 [SMILES: *c1ccc(Cl)c(Cl)c1] | JC-917 |
| H₂N(CH₂)₂ | H | CH₃ | H | H | 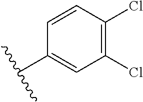 [SMILES: *c1ccc(Cl)c(Cl)c1] | JC-918 (TFA salt) |
| 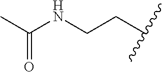 [SMILES: *CCNC(C)=O] | H | CH₃ | H | H | 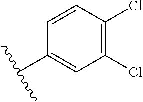 [SMILES: *c1ccc(Cl)c(Cl)c1] | JC-919 |
| 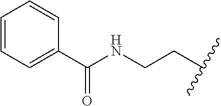 [SMILES: *CCNC(=O)c1ccccc1] | H | CH₃ | H | H | 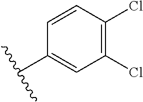 [SMILES: *c1ccc(Cl)c(Cl)c1] | JC-920 |
| 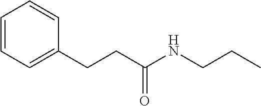 | H | CH₃ | H | H | 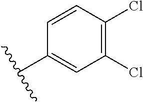 [SMILES: *c1ccc(Cl)c(Cl)c1] | JC-921 |
| 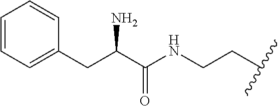 [SMILES: *CCNC(=O)[C@H](N)Cc1ccccc1] | H | CH₃ | H | H | 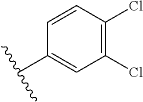 [SMILES: *c1ccc(Cl)c(Cl)c1] | JC-922 |
| 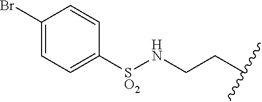 [SMILES: *CCNS(=O)(=O)c1ccc(Br)cc1] | H | CH₃ | H | H | 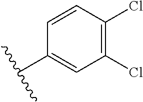 [SMILES: *c1ccc(Cl)c(Cl)c1] | JC-923 |
| Boc—(NH)—(CH₂)₃ | H | CH₃ | H | H | 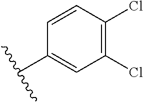 [SMILES: *c1ccc(Cl)c(Cl)c1] | JC-932 |
| H₂N(CH₂)₃ | H | CH₃ | H | H | 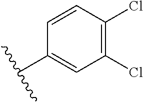 [SMILES: *c1ccc(Cl)c(Cl)c1] | JC-933 (TFA salt) |
| 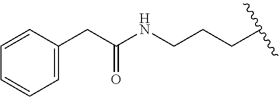 [SMILES: *CCCNC(=O)Cc1ccccc1] | H | CH₃ | H | H | 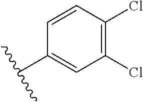 [SMILES: *c1ccc(Cl)c(Cl)c1] | JC-934 |
| 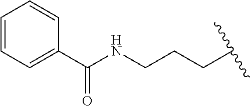 [SMILES: *CCCNC(=O)c1ccccc1] | H | CH₃ | H | H | 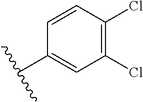 [SMILES: *c1ccc(Cl)c(Cl)c1] | JC-935 |
| 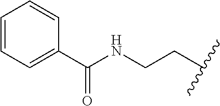 [SMILES: *CCNC(=O)c1ccccc1] | H | CH₃ | H | H | 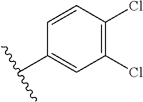 [SMILES: *c1ccc(Cl)c(Cl)c1] | JC-952 |
| 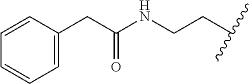 [SMILES: *CCNC(=O)Cc1ccccc1] | H | CH₃ | H | H | 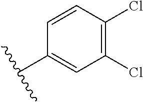 [SMILES: *c1ccc(Cl)c(Cl)c1] | JC-953 |
| 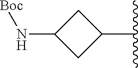 [SMILES: *C1CC(NC(=O)OC(C)(C)C)C1] | H | CH₃ | H | H | 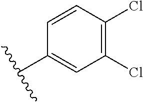 [SMILES: *c1ccc(Cl)c(Cl)c1] | JC-956 |
| 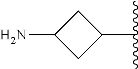 [SMILES: *C1CC(N)C1] | H | CH₃ | H | H | 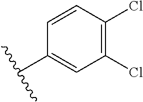 [SMILES: *c1ccc(Cl)c(Cl)c1] | JC-957 |
| 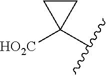 [SMILES: *C1(C(=O)O)CC1] | H | CH₃ | H | H | 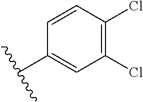 [SMILES: *c1ccc(Cl)c(Cl)c1] | JC-900 |
| 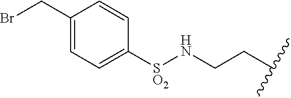 [SMILES: *CCNS(=O)(=O)c1ccc(CBr)cc1] | H | CH₃ | H | H | 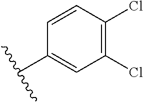 [SMILES: *c1ccc(Cl)c(Cl)c1] | JC-993 |
| 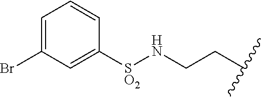 [SMILES: *CCNS(=O)(=O)c1cccc(Br)c1] | H | CH₃ | H | H | 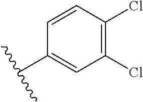 [SMILES: *c1ccc(Cl)c(Cl)c1] | JC-994 |
| 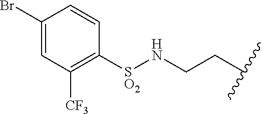 [SMILES: *CCNS(=O)(=O)c1ccc(Br)cc1C(F)(F)F] | H | CH₃ | H | H | 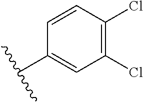 [SMILES: *c1ccc(Cl)c(Cl)c1] | JC-995 |
| 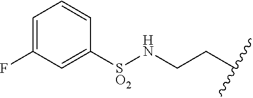 [SMILES: *CCNS(=O)(=O)c1cccc(F)c1] | H | CH₃ | H | H | 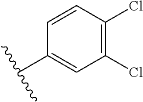 [SMILES: *c1ccc(Cl)c(Cl)c1] | JC-996 |
| 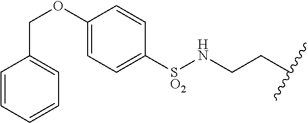 [SMILES: *CCNS(=O)(=O)c1ccc(OCc2ccccc2)cc1] | H | CH₃ | H | H | 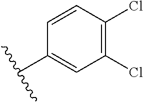 [SMILES: *c1ccc(Cl)c(Cl)c1] | JC-997 |
| 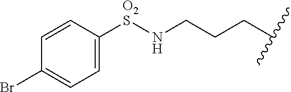 [SMILES: *CCCNS(=O)(=O)c1ccc(Br)cc1] | H | CH₃ | H | H | 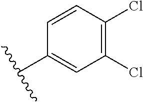 [SMILES: *c1ccc(Cl)c(Cl)c1] | JC-998 |
| 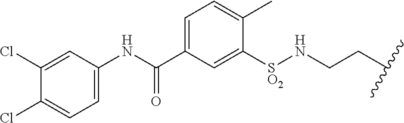 [SMILES: *CCNS(=O)(=O)c1cc(C(=O)Nc2ccc(Cl)c(Cl)c2)ccc1C] | H | CH₃ | H | H | 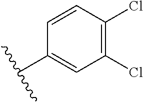 [SMILES: *c1ccc(Cl)c(Cl)c1] | JC- 1021 |
| 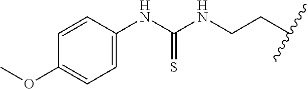 [SMILES: *CCNC(=S)Nc1ccc(OC)cc1] | H | CH₃ | H | H | 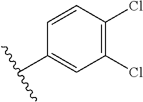 [SMILES: *c1ccc(Cl)c(Cl)c1] | JC-999 |
| 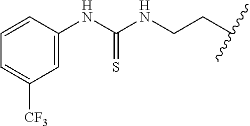 [SMILES: *CCNC(=S)Nc1cccc(C(F)(F)F)c1] | H | CH₃ | H | H | 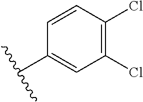 [SMILES: *c1ccc(Cl)c(Cl)c1] | JC- 1000 |
| 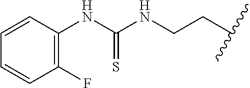 [SMILES: *CCNC(=S)Nc1ccccc1F] | H | CH₃ | H | H | 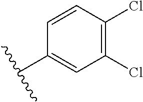 [SMILES: *c1ccc(Cl)c(Cl)c1] | JC- 1001 |
| 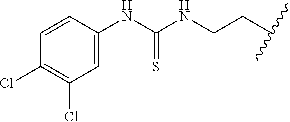 [SMILES: *CCNC(=S)Nc1ccc(Cl)c(Cl)c1] | H | CH₃ | H | H | 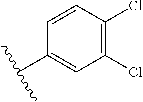 [SMILES: *c1ccc(Cl)c(Cl)c1] | JC- 1002 |
| 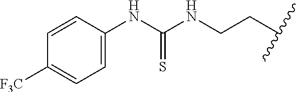 [SMILES: *CCNC(=S)Nc1ccc(C(F)(F)F)cc1] | H | CH₃ | H | H | 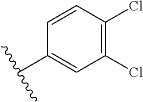 [SMILES: *c1ccc(Cl)c(Cl)c1] | JC- 1003 |
| 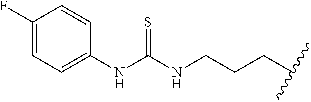 [SMILES: *CCCNC(=S)Nc1ccc(F)cc1] | H | CH₃ | H | H | 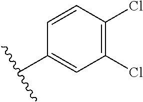 [SMILES: *c1ccc(Cl)c(Cl)c1] | JC- 1015 |

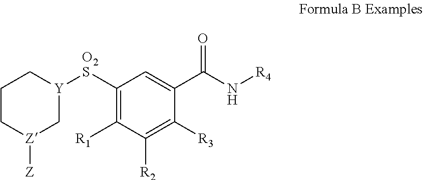

TABLE 2
Formula B examples

| Z | Z′ | Y | R₁ | R₂ | R₃ | R₄ | ID |
|---|---|---|---|---|---|---|---|
| NH—Boc | CH | N | CH₃ | H | H | 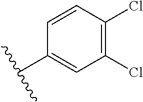 [SMILES: *c1ccc(Cl)c(Cl)c1] | JC-928 |
| NH₂ | CH | N | CH₃ | H | H | 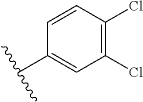 [SMILES: *c1ccc(Cl)c(Cl)c1] | JC-929 (TFA salt) |
| Boc | N | CH | CH₃ | H | H | 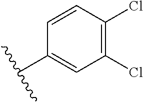 [SMILES: *c1ccc(Cl)c(Cl)c1] | JC-950 |
| H | N | CH | CH₃ | H | H | 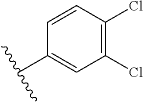 [SMILES: *c1ccc(Cl)c(Cl)c1] | JC-951 (TFA salt) |
| BocHN | CH | CH | CH₃ | H | H | 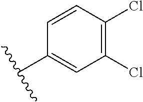 [SMILES: *c1ccc(Cl)c(Cl)c1] | JC-954 |
| H₂N | CH | CH | CH₃ | H | H | 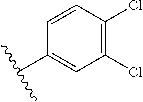 [SMILES: *c1ccc(Cl)c(Cl)c1] | JC-955 |
| 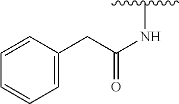 [SMILES: *NC(=O)Cc1ccccc1] | CH | N | CH₃ | H | H | 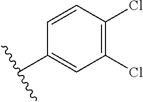 [SMILES: *c1ccc(Cl)c(Cl)c1] | JC-930 |
| 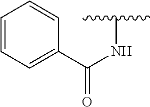 [SMILES: *NC(=O)c1ccccc1] | CH | N | CH₃ | H | H | 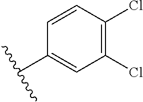 [SMILES: *c1ccc(Cl)c(Cl)c1] | JC-931 |
| 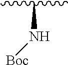 | CH | N | CH₃ | H | H | 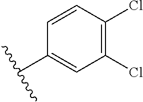 [SMILES: *c1ccc(Cl)c(Cl)c1] | JC-958 |
| 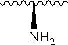 | CH | N | CH₃ | H | H | 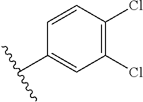 [SMILES: *c1ccc(Cl)c(Cl)c1] | JC-959 (TFA salt) |
| 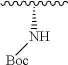 | CH | N | CH₃ | H | H | 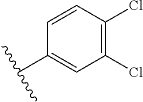 [SMILES: *c1ccc(Cl)c(Cl)c1] | JC-962 |
| 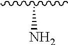 | CH | N | CH₃ | H | H | 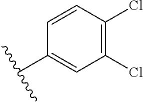 [SMILES: *c1ccc(Cl)c(Cl)c1] | JC-963 (TFA salt) |
| 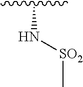 | CH | N | CH₃ | H | H | 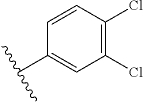 [SMILES: *c1ccc(Cl)c(Cl)c1] | JC-964 |
| 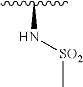 | CH | N | CH₃ | H | H | 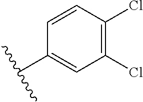 [SMILES: *c1ccc(Cl)c(Cl)c1] | JC-960 |
| 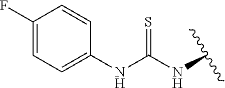 | CH | N | CH₃ | H | H | 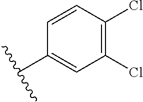 [SMILES: *c1ccc(Cl)c(Cl)c1] | JC-965 |
| 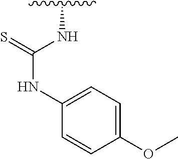 | CH | N | CH₃ | H | H | 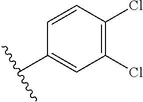 [SMILES: *c1ccc(Cl)c(Cl)c1] | JC- 1004 |
| 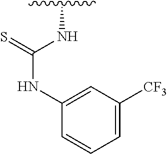 | CH | N | CH₃ | H | H | 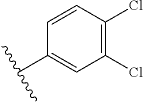 [SMILES: *c1ccc(Cl)c(Cl)c1] | JC- 1005 |
| 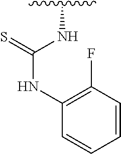 | CH | N | CH₃ | H | H | 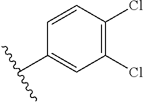 [SMILES: *c1ccc(Cl)c(Cl)c1] | JC- 1006 |
| 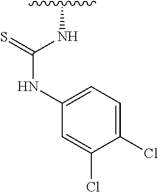 | CH | N | CH₃ | H | H | 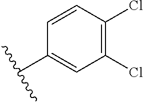 [SMILES: *c1ccc(Cl)c(Cl)c1] | JC- 1007 |
| 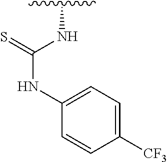 | CH | N | CH₃ | H | H | 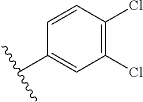 [SMILES: *c1ccc(Cl)c(Cl)c1] | JC- 1008 |

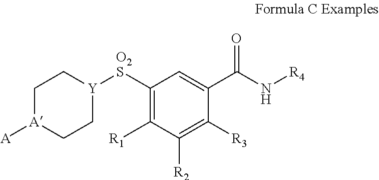

TABLE 3
Formula C examples

| A | A′ | Y | R₁ | R₂ | R₃ | R₄ | ID |
|---|---|---|---|---|---|---|---|
| 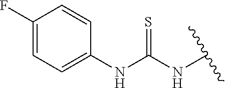 [SMILES: *NC(=S)Nc1ccc(F)cc1] | CH | N | CH₃ | H | H | 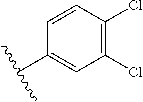 [SMILES: *c1ccc(Cl)c(Cl)c1] | JC- 970 |
| 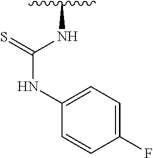 | CH | N | CH₃ | H | H | 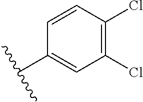 [SMILES: *c1ccc(Cl)c(Cl)c1] | JC- 961 |
| 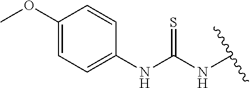 [SMILES: *NC(=S)Nc1ccc(OC)cc1] | CH | N | CH₃ | H | H | 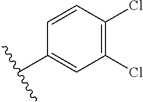 [SMILES: *c1ccc(Cl)c(Cl)c1] | JC- 1016 |
| 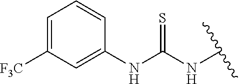 [SMILES: *NC(=S)Nc1cccc(C(F)(F)F)c1] | CH | N | CH₃ | H | H | 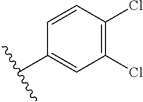 [SMILES: *c1ccc(Cl)c(Cl)c1] | JC- 1017 |
| 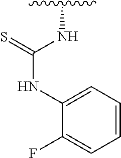 | CH | N | CH₃ | H | H | 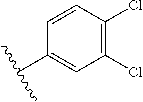 [SMILES: *c1ccc(Cl)c(Cl)c1] | JC- 1018 |
| 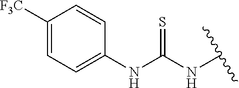 [SMILES: *NC(=S)Nc1ccc(C(F)(F)F)cc1] | CH | N | CH₃ | H | H | 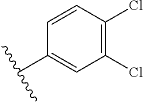 [SMILES: *c1ccc(Cl)c(Cl)c1] | JC- 1020 |
| 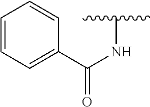 [SMILES: *NC(=O)c1ccccc1] | CH | N | CH₃ | H | H | 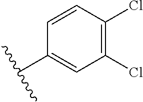 [SMILES: *c1ccc(Cl)c(Cl)c1] | JC-966 |
| 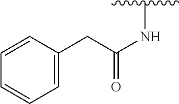 [SMILES: *NC(=O)Cc1ccccc1] | CH | N | CH₃ | H | H | 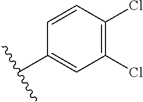 [SMILES: *c1ccc(Cl)c(Cl)c1] | JC-967 |
| 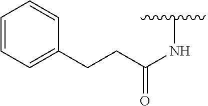 [SMILES: *NC(=O)CCc1ccccc1] | CH | N | CH₃ | H | H | 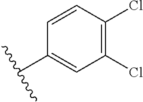 [SMILES: *c1ccc(Cl)c(Cl)c1] | JC-968 |
| 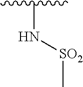 [SMILES: *NS(C)(=O)=O] | CH | N | CH₃ | H | H | 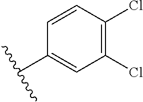 [SMILES: *c1ccc(Cl)c(Cl)c1] | JC-969 |
| 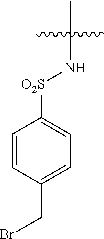 [SMILES: *NS(=O)(=O)c1ccc(CBr)cc1] | CH | N | CH₃ | H | H | 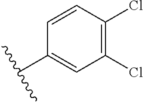 [SMILES: *c1ccc(Cl)c(Cl)c1] | JC- 1009 |
| 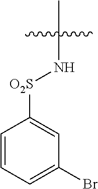 [SMILES: *NS(=O)(=O)c1cccc(Br)c1] | CH | N | CH₃ | H | H | 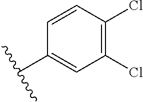 [SMILES: *c1ccc(Cl)c(Cl)c1] | JC- 1010 |
| 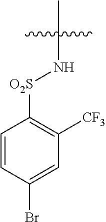 [SMILES: *NS(=O)(=O)c1ccc(Br)cc1C(F)(F)F] | CH | N | CH₃ | H | H | 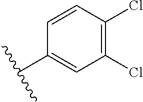 [SMILES: *c1ccc(Cl)c(Cl)c1] | JC- 1011 |
| 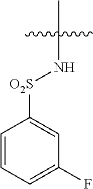 [SMILES: *NS(=O)(=O)c1cccc(F)c1] | CH | N | CH₃ | H | H | 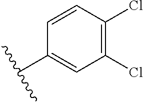 [SMILES: *c1ccc(Cl)c(Cl)c1] | JC- 1012 |
| 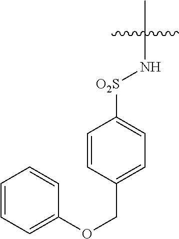 [SMILES: *NS(=O)(=O)c1ccc(COc2ccccc2)cc1] | CH | N | CH₃ | H | H | 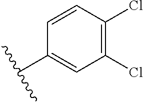 [SMILES: *c1ccc(Cl)c(Cl)c1] | JC- 1013 |
| 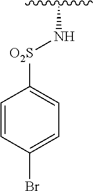 | CH | N | CH₃ | H | H | 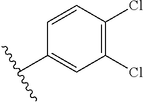 [SMILES: *c1ccc(Cl)c(Cl)c1] | JC- 1014 |

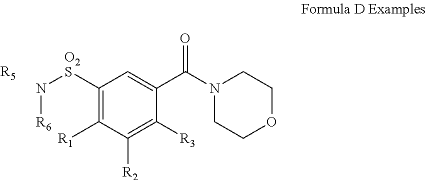

TABLE 4
Formula D examples

| R₅ | R₆ | R₁ | R₂ | R₃ | ID |
|---|---|---|---|---|---|
| 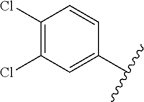 [SMILES: *c1ccc(Cl)c(Cl)c1] | H | CH₃ | H | H | JC-867 |

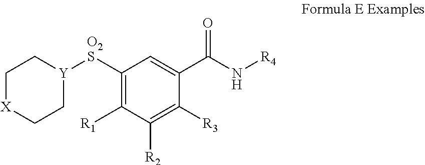

TABLE 5
Formula E Examples

| X | Y | R₁ | R₂ | R₃ | R₄ | ID |
|---|---|---|---|---|---|---|
| O | N | CH₃ | H | H | 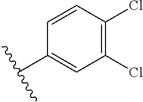 [SMILES: *c1ccc(Cl)c(Cl)c1] | JC-885 |
| CF₂ | N | CH₃ | H | H | 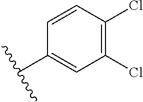 [SMILES: *c1ccc(Cl)c(Cl)c1] | JC-886 |
| CH—NH— Boc | N | CH₃ | H | H | 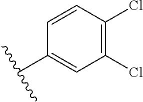 [SMILES: *c1ccc(Cl)c(Cl)c1] | JC-896 |
| CH—NH₂ | N | CH₃ | H | H | 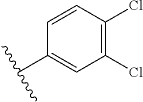 [SMILES: *c1ccc(Cl)c(Cl)c1] | JC-906 (TFA salt) |
| N-Boc | CH | CH₃ | H | H | 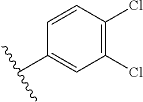 [SMILES: *c1ccc(Cl)c(Cl)c1] | JC-910 |
| NH | CH | CH₃ | H | H | 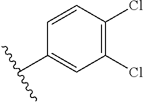 [SMILES: *c1ccc(Cl)c(Cl)c1] | JC-911 (TFA salt) |
| NCH₃ | N | CH₃ | H | H | 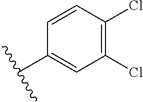 [SMILES: *c1ccc(Cl)c(Cl)c1] | JC- 916 |
| CH—NH—Boc | CH | CH₃ | H | H | 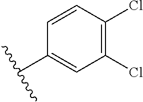 [SMILES: *c1ccc(Cl)c(Cl)c1] | JC- 924 |
| CH—NH₂ | CH | CH₃ | H | H | 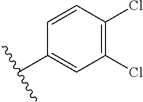 [SMILES: *c1ccc(Cl)c(Cl)c1] | JC- 925 |
| 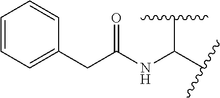 [SMILES: *C(*)NC(=O)Cc1ccccc1] | CH | CH₃ | H | H | 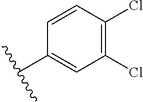 [SMILES: *c1ccc(Cl)c(Cl)c1] | JC- 926 |
| 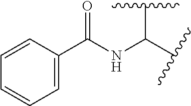 [SMILES: *C(*)NC(=O)c1ccccc1] | CH | CH₃ | H | H | 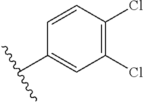 [SMILES: *c1ccc(Cl)c(Cl)c1] | JC- 927 |

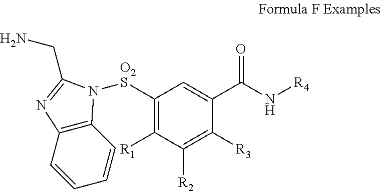

TABLE 6
Formula F Examples

| R₁ | R₂ | R₃ | R₄ | ID |
|---|---|---|---|---|
| CH₃ | H | H | 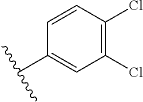 [SMILES: *c1ccc(Cl)c(Cl)c1] | JC-904 (TFA salt) |

Inhibitory data is shown in Table 7. Trypanosoma brucei cytotoxicity of JC-229 and analogs were measured using ALAMARBLUE® assay (Rampersad, S. N. Multiple applications of ALAMARBLUE® as an indicator of metabolic function and cellular health in cell viability bioassays. Sensors (Basel) 12, 12347-12360 (2012). Trypanosome cells were seeded at 1×10³ cells/ml density in 180 μl in a 96-well culture plate in triplicate. These cells were incubated with indicated concentration of JC compounds for 72 hours. DMSO was used as a vehicle control at 0.0075%. JC compound-treated cultures were then incubated with ALAMARBLUE® dye for 4-6 hours and percentage of live cells was determined using Synergy H1 microplate reader (BioTek) with 560/590 nm (excitation/emission) filter settings and Relative Fluorescence Unit (RFU) values were obtained. The percentage of viable cells were calculated from the RFU values by normalizing to DMSO treated control. In one embodiment, the inhibitor is characterized in that it provides at least 50%, at least 80% or at least 90% inhibition at a concentration of 5 μM when tested in accordance with the above conditions.

TABLE 7
% Inhibition

| ID | @10 μM | @5 μM | @2 μM |
|---|---|---|---|
| JC-229 | 100 | 76 | 24 |
| JC-853 | 81 | | −17 |
| JC-854 | 14 | | −27 |
| JC-855 | 89 | | −5 |
| JC-856 | 22 | | −7 |
| JC-857 | 14 | | −5 |
| JC-858 | 100 | 86 | 13 |
| JC-859 | 9 | | 13 |
| JC-860 | 24 | | 5 |
| JC-861 | 4 | | 5 |
| JC-862 | 27 | | −19 |
| JC-863 | 2 | | −40 |
| JC-864 | 42 | | −20 |
| JC-865 | 55 | | −1 |
| JC-866 | 23 | | −15 |
| JC-867 | 31 | | −14 |
| JC-868 | 20 | | 1 |
| JC-869 | 30 | | −34 |
| JC-870 | 49 | | −34 |
| JC-871 | 79 | | −3 |
| JC-872 | 21 | | −13 |
| JC-873 | 100 | 1 | −4 |
| JC-874 | 97 | 51.8 | 1 |
| JC-885 | 38 | | 13 |
| JC-886 | 100 | 93.7 | 3 |
| JC-887 | 14 | | −9 |
| JC-888 | 100 | 62.1 | 16 |
| JC-889 | −11 | | −3 |
| JC-890 | 37 | | 4 |
| JC-891 | 100 | 88.8 | 13 |
| JC-892 | 1 | | 13 |
| JC-893 | −5 | | 2 |
| JC-894 | 2 | | −4 |
| JC-895 | 45 | | 10 |
| JC-896 | 92 | 69.4 | 17 |
| JC-897 | 36 | | 0 |
| JC-898 | 57 | | 6 |
| JC-899 | 14 | | 3 |
| JC-900 | 6 | | 17 |
| JC-901 | 94 | 14.7 | 15 |
| JC-902 | 56 | | 14 |
| JC-903 | 37 | | 11 |
| JC-904 | 2 | | 14 |
| JC-905 | 10 | | 8 |
| JC-906 | 100 | 100 | 23 |
| JC-907 | 5 | | 12 |
| JC-908 | 62 | | 18 |
| JC-909 | 17 | | 15 |
| JC-910 | 73 | | 18 |
| JC-911 | 25 | | 11 |
| JC-912 | 58 | | −5 |
| JC-913 | 6 | | 2 |
| JC-914 | −3 | | −7 |
| JC-915 | 6 | | −11 |
| JC-916 | 36 | | −2 |
| JC-917 | 73 | | −14 |
| JC-918 | 56 | | −20 |
| JC-919 | 18 | | −10 |
| JC-920 | −9 | | −5 |
| JC-921 | 100 | 60.5 | −11 |
| JC-922 | 19 | | −7 |
| JC-923 | 100 | 100.9 | 2 |
| JC-924 | 100 | 99.4 | −2 |
| JC-925 | 17 | | −8 |
| JC-926 | 100 | 28.9 | −8 |
| JC-927 | 100 | 42.4 | 0 |
| JC-928 | 100 | 99.6 | 15 |
| JC-929 | 100 | 99.7 | 5 |
| JC-930 | 100 | 99.7 | 4 |
| JC-931 | 100 | 98.5 | 3 |
| JC-932 | 73 | | 7 |
| JC-933 | 60 | | 5 |
| JC-934 | 34 | | 11 |
| JC-935 | 19 | | 3 |
| Jc-950 | | 3 | 28 |
| JC-951 | | 100 | 29 |
| JC-952 | | 4 | −62 |
| JC-953 | | 32 | −34 |
| JC-954 | | 35 | 10 |
| JC-955 | | 100 | 25 |
| JC-956 | | 19 | −32 |
| JC-957 | | 84 | −30 |
| JC-958 | | 100 | 53 |
| JC-959 | | 100 | 64 |
| JC-960 | | −17 | −14 |
| JC-961 | | 100 | 100 |
| JC-962 | | 100 | 36 |
| JC-963 | | 100 | 96 |
| JC-964 | | −23 | −16 |
| JC-965 | | 100 | 64 |
| JC-966 | | 0 | 13 |
| JC-967 | | −24 | 15 |
| JC-968 | | 5 | 1 |
| JC-969 | | −4 | 18 |
| JC-970 | | 100 | 100 |
| JC-993 | | 100 | 76 |
| JC-994 | | 61 | −17 |
| JC-995 | | 93 | −34 |
| JC-996 | | 69 | −19 |
| JC-997 | | 92 | −9 |
| JC-998 | | 100 | 0 |
| JC-999 | | −6 | −7 |
| JC-1000 | | 58 | 21 |
| JC-1001 | | 66 | 49 |
| JC-1002 | | 100 | 34 |
| JC-1003 | | 80 | 0 |
| JC-1004 | | 94 | 32 |
| JC-1005 | | 100 | 100 |
| JC-1006 | | 101 | 17 |
| JC-1007 | | 101 | 100 |
| JC-1008 | | 100 | 100 |
| JC-1009 | | 100 | 99 |
| JC-1010 | | −15 | 9 |
| JC-1011 | | 101 | 63 |
| JC-1012 | | 97 | 36 |
| JC-1013 | | −9 | 13 |
| JC-1014 | | 100 | 100 |
| JC-1015 | | 100 | 11 |
| JC-1016 | | 100 | 52 |
| JC-1017 | | 100 | 100 |
| JC-1018 | | 100 | 100 |
| JC-1019 | | 100 | 8 |
| JC-1020 | | 100 | 100 |
| JC-1021 | | −5 | 0 |

In one embodiment, a method is provided for treating a patient in need of treatment for a Trypanosoma brucei infection. Any one of the inhibitors disclosed in this specification may be administered. In one embodiment, the inhibitor is selected such that it provides at least 50%, at least 80% or at least 90% inhibition at a concentration of 5 μM when tested in accordance with the in vitro conditions specified elsewhere in this disclosure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition of matter with a structure as recited in Formula C-2: 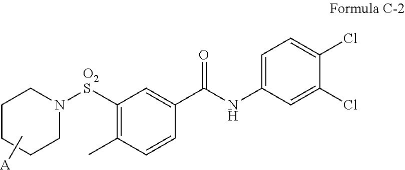
wherein A is selected from H, NH₂; NH-Boc (tert-butyloxycarbonyl); NH(SO₂)CH₃ and: 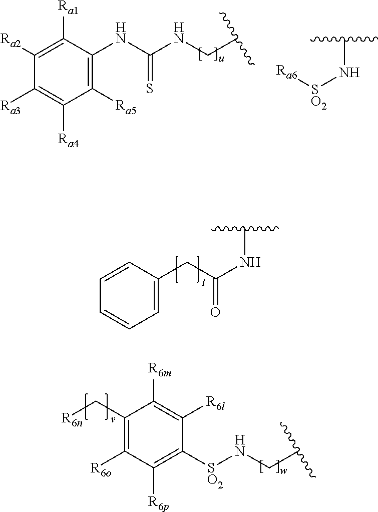
wherein t is 0, 1, 2 or 3; u is 0, 1, 2 or 3; Rₐ₁, Rₐ₂, Rₐ₃, Rₐ₄, Rₐ₅ are independently selected from H, F, Cl, OCH₃ and CF₃; Rₐ₆ is methyl, ethyl or propyl; v is 0, 1, 2 or 3; w is an integer from 0-6, inclusive; R₆ₗ, R₆ₘ, R₆ₙ, R₆ₒ and R₆ₚ are independently selected from H, F, Br, CH₃, CF₃ OBz and (CO)NPh where Ph is a 3,4-dichlorophenyl.

2. A method of treating a patient, the method comprising:
administering the composition of matter as recited in claim 1 to a patient for treatment of a Trypanosoma brucei infection.

3. The composition of matter as recited in claim 1, wherein the structure is:

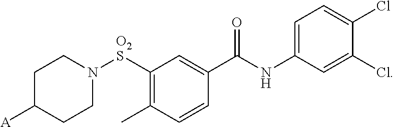

4. The composition of matter as recited in claim 3, wherein the structure is: 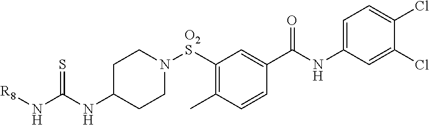
where R₈ is selected from:

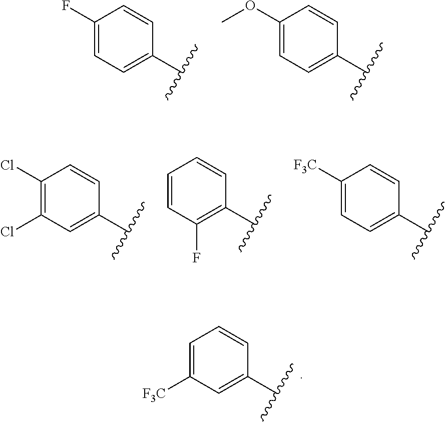

5. The composition of matter as recited in claim 3, where in the structure is:

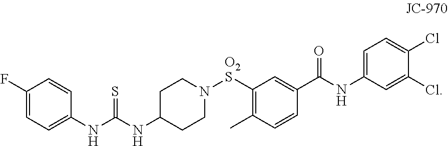 [SMILES: Cc1ccc(C(=O)Nc2ccc(Cl)c(Cl)c2)cc1S(=O)(=O)N1CCC(NC(=S)Nc2ccc(F)cc2)CC1]

6. The composition of matter as recited in claim 1, wherein the structure is:

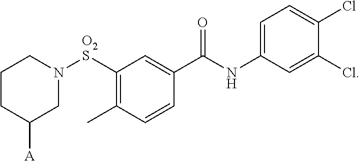

7. The composition as recited in claim 6, wherein the structure is:

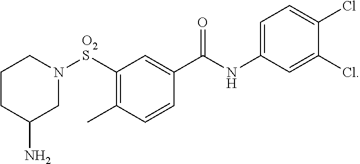 [SMILES: Cc1ccc(C(=O)Nc2ccc(Cl)c(Cl)c2)cc1S(=O)(=O)N1CCCC(N)C1]

8. The composition as recited in claim 6, wherein the structure is:

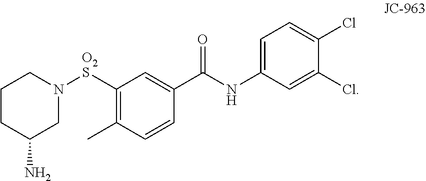 [SMILES: Cc1ccc(C(=O)Nc2ccc(Cl)c(Cl)c2)cc1S(=O)(=O)N1CCC[C@@H](N)C1]

9. The composition as recited in claim 1, wherein the composition inhibits Trypanosoma brucei by at least 50% at 10 μM but inhibits HEK293 by less than 20% at 10 μM.

10. A composition of matter with a structure as recited in Formula B: 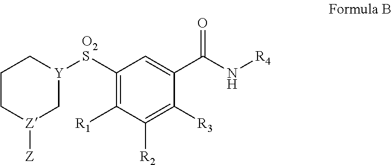
wherein R₁, R₂ and R₃ are independently selected from H and a C1-C3 alkyl;
R₄ is an organic group selected from: 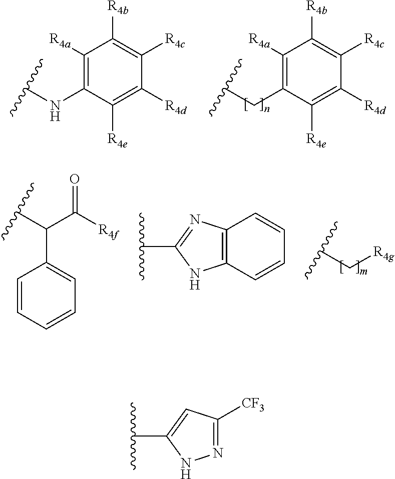
wherein R₄ₐ, R_{4b}, R_{4c}, R_{4d} and R₄ₑ are independently selected from H and Cl, n is 0, 1, 2 or 3, R_{4f} is NH₂, N(CH₃)₂, CO₂H, CO₂CH₃, R_{4g} is NH₂, N(CH₃)₂, OH and m is 0, 1, 2 or 3;
Y is N or CH;
Z′ is Nor CH; and
Z is selected from H, NH₂; NH-Boc (tert-butyloxycarbonyl); NH(SO₂)CH₃, a structure selected from 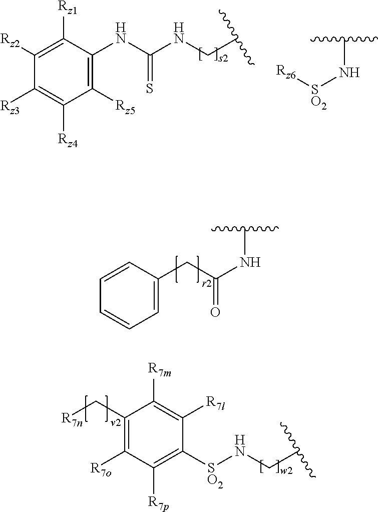
wherein r2 is 0, 1, 2 or 3; s2 is 0, 1, 2 or 3 and R_{z1}, R_{z2}, R_{z3}, R_{z4}, R_{z5} are independently selected from H, F, Cl, OCH₃ and CF₃; R_{z6} is methyl, ethyl or propyl; v2 is 0, 1, 2 or 3; w2 is an integer from 0-6 (inclusive); R₇ₗ, R₇ₘ, R₇ₙ, R₇ₒ and R₇ₚ are independently selected from H, F, Br, CH₃, CF₃, OBz and (CO)NPh where Ph is a 3,4-dichlorophenyl;
or a pharmaceutically acceptable salt thereof.

11. The composition as recited in claim 10, where R₁ is methyl, R₂ is H and R₃ is H.

12. The composition as recited in claim 11, wherein Y is N.

13. The composition as recited in claim 12, wherein R₄ is 3,4-dichlorophenyl such that the structure is:

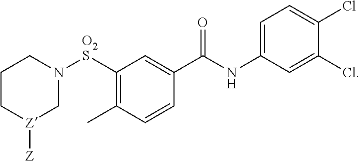

14. A method of treating a patient, the method comprising:
administering the composition of matter as recited in claim 10 to a patient for treatment of a Trypanosoma brucei infection.

15. The composition as recited in claim 10, wherein the composition inhibits Trypanosoma brucei by at least 50% at 10 μM but inhibits HEK293 by less than 20% at 10 μM.

16. A composition of matter with a structure as recited in Formula C: 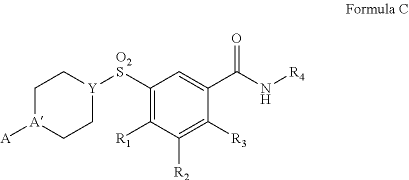
wherein R₁, R₂ and R₃ are independently selected from H and a C1-C3 alkyl;
R₄ is an organic group selected from: 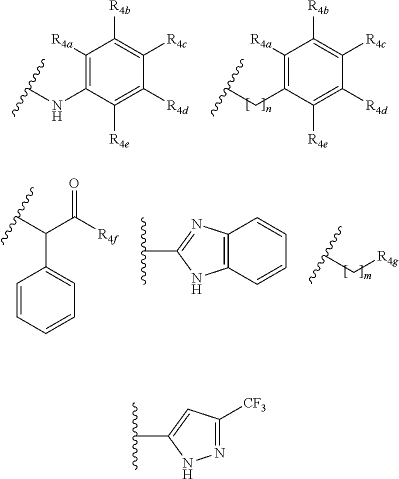
wherein R₄ₐ, R_{4b}, R_{4c}, R_{4d} and R₄ₑ are independently selected from H and Cl, n is 0, 1, 2 or 3, R_{4f} is NH₂, N(CH₃)₂, CO₂H, CO₂CH₃, R_{4g} is NH₂, N(CH₃)₂, OH and m is 0, 1, 2 or 3;
Y is N or CH;
A′ is N or CH; and
A is selected from H, NH₂; NH-Boc (tert-butyloxycarbonyl); NH(SO₂)CH₃, a structure selected from 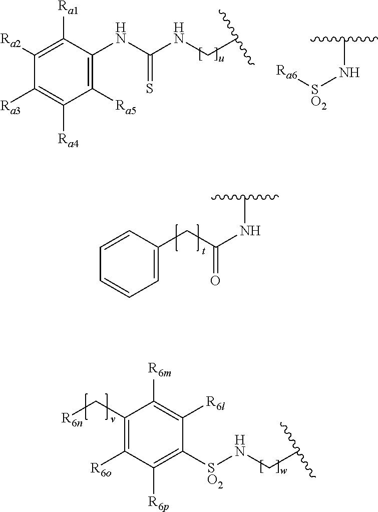
wherein t is 0, 1, 2 or 3; u is 0, 1, 2 or 3 and Rₐ₁, Rₐ₂, Rₐ₃, Rₐ₄, Rₐ₅ are independently selected from H, F, Cl, OCH₃ and CF₃; Rₐ₆ is methyl, ethyl or propyl; v is 0, 1, 2 or 3; w is an integer from 0-6 (inclusive); R₆ₗ, R₆ₘ, R₆ₙ, R₆ₒ and R₆ₚ are independently selected from H, F, Br, CH₃, CF₃ OBz and (CO)NPh where Ph is a 3,4-dichlorophenyl;
or a pharmaceutically acceptable salt thereof.

17. The composition as recited in claim 16, where R₁ is methyl, R₂ is H and R₃ is H.

18. The composition as recited in claim 17, wherein Y is N.

19. A method of treating a patient, the method comprising:
administering the composition of matter as recited in claim 16 to a patient for treatment of a Trypanosoma brucei infection.

20. The composition as recited in claim 16, wherein the composition inhibits Trypanosoma brucei by at least 50% at 10 μM but inhibits HEK293 by less than 20% at 10 μM.
